(19) United States
(12) Patent Application Publication
(10) Pub. No.: US 2012/0009203 A1
(43) Pub. Date: Jan. 12, 2012

(54) Depletion of cancer stem cells

(76) Inventor: Anahid Jewett, Valencia, CA (US)

(21) Appl. No.: 13/135,222

(22) Filed: Jun. 29, 2011

Related U.S. Application Data
(60) Provisional application No. 61/398,681, filed on Jun. 29, 2010.

(51) Int. Cl. A61K 35/26; A61P 37/06; A61P 35/00; A61K 31/337; A61K 39/395

(52) U.S. Cl. 424/172.1; 424/93.7; 514/449

(57) ABSTRACT

Compositions and methods are provided for killing of cancer stem cells, and for the transplantation of pluripotent stem cells and differentiated cells derived therefrom.

### BACKGROUND OF THE INVENTION

Immunosuppression and tumor escape from immune recognition are thought to be major factors responsible for the establishment and progression of cancer. A number of factors responsible for the suppression of NK cell cytotoxicity in humans have been identified previously. However, the significance and the precise mechanism of this suppression induced during the interaction of NK cells with either tumor cells or healthy primary cells are not well understood. It is shown that freshly isolated tumor infiltrating NK cells are not cytotoxic to autologous tumors. Moreover, NK cells obtained from the peripheral blood of patients with cancer have significantly reduced cytotoxic activity. In addition, NK cell cytotoxicity is suppressed after their interaction with stem cells. However, interaction of NK cells with the resistant tumors does not lead to suppression of NK cell cytotoxicity.

Many mechanisms have been proposed for the functional inactivation of tumor associated NK cells including the over-expression of Fas ligand, the loss of mRNA for granzyme B and decreased CD16 and its associated zeta chain.

Many metastatic tumor cells exhibit constitutively elevated NFκB activity. Increased NFκB activity is shown to have a causal relationship to neoplastic transformation, and uncontrolled cell growth in many cell types. Human solid tumors exhibit constitutively activated NFκB.

We have previously shown that NK resistant primary oral keratinocyte tumors demonstrate higher nuclear NFκB activity and secrete significant levels of Granulocyte Monocyte-Colony Stimulating Factor (GM-CSF), Interleukin(IL)-1β, IL-6 and IL-8. Moreover, the addition of Non-steroidal anti-inflammatory drugs (NSAIDs) which inhibit NFκB have the ability to reverse immunosuppression induced by a tobacco-specific carcinogen in addition to their well-established ability to decrease oral dysplasia as well as induction of overt cancer in transgenic animals. In agreement, we have previously demonstrated that inhibition of NFκB by Sulindac treatment of tumor cells increases functional activity of NK cells. In addition, targeted inhibition of NFκB in skin epithelial cells resulted in the induction of auto-immunity and inflammation.

The exact mechanism by which NFκB nuclear function in oral keratinocytes modulate and shape the function of key interacting immune effectors is yet to be determined. We have previously shown that inhibition of NFκB by the IκB super-repressor in HEp2 tumors leads to significant increase in cytotoxicity and secretion of IFN-γ by the human NK cells. However, neither the underlying significance nor the physiological relevance of NFκB modulation in tumors or in primary cells responsible for the alteration of NK cell cytotoxic function have been addressed or studied previously. It is clear that the objective in cancer is to enhance the function of cytotoxic immune effectors and in auto-immunity and inflammation the aim is to inhibit immune effector function. Therefore, dissection of the underlying mechanisms of immune activation when NFκB is modulated in the cells might help design strategies to target each disease accordingly. Indeed, targeted inhibition of NFκB function in both the intestinal epithelial cells and the myeloid cells was previously shown to result in a significant decrease in the size and the numbers of the tumor cells.

Regenerative medicine is the process of creating living, functional tissues to repair or replace tissue or organ function lost due to age, disease, damage, or congenital defects. This field holds the promise of regenerating damaged tissues and organs in the body by introducing outside cells, tissue, or even whole organs to integrate and become a part of tissues or replace whole organ. Importantly, regenerative medicine has the potential to solve the problem of the shortage of organs available for donation compared to the number of patients that require life-saving organ transplantation.

One key to the success of regenerative medicine strategies has been the ability to isolate and generate stem cells, including pluripotent stem cells. In one aspect, pluripotent stem cells can be differentiated into a necessary cell type, where the mature cells are used to replace tissue that is damaged by disease or injury. This type of treatment could be used to replace neurons damaged by spinal cord injury, stroke, Alzheimer's disease, Parkinson's disease, or other neurological problems. Cells grown to produce insulin could treat people with diabetes and heart muscle cells could repair damage after a heart attack. This list could conceivably include any tissue that is injured or diseased.

The generation of pluripotent stem cells that are genetically identical to an individual provides unique opportunities for basic research and for potential immunologically-compatible novel cell-based therapies. Methods to reprogram primate somatic cells to a pluripotent state include differentiated somatic cell nuclear transfer, differentiated somatic cell fusion with pluripotent stem cells, and direct reprogramming to produce induced pluripotent stem cells (iPS cells) (Takahashi K, et al. (2007) Cell 131:861-872; Park I H, et al. (2008) Nature 451:141-146; Yu J, et al. (2007) Science 318:1917-1920; Kim D, et al. (2009) Cell Stem Cell 4:472-476; Soldner F, et al. (2009) Cell. 136:964-977; Huangfu D, et al. (2008) Nature Biotechnology 26:1269-1275; Li W, et al. (2009) Cell Stem Cell 4:16-19).

A significant first hurdle in stem cell-based therapy is the differentiation of pluripotent cells into a desired tissue type. Such methods currently rely on the step-wise introduction of factors and conditions to guide the cells down a developmental pathway, resulting eventually in a mature or committed progenitor cell that can transplanted into a patient.

Embryonic stem cells (ESCs) are an attractive source for tissue regeneration and repair therapies because they can be cultured indefinitely in vitro and can be differentiated into virtually any cell type in the adult body. However, for this approach to succeed, the transplanted ESCs must engraft successfully and survive long enough to permit a therapeutic benefit. An important obstacle facing the engraftment and function of hESCs is transplant rejection by the immune system. The present invention addresses this issue.

### SUMMARY OF THE INVENTION

Compositions and methods are provided for treatment of cancer, particularly the killing of cancer stem cells, by administration of natural killer cells. Alternative, the combination of a taxane and NAC may be used in the absence of NK cells to kill cancer stem cells. In some embodiments the cancer stem cells are carcinoma stem cells, including without limitation squamous carcinoma stem cells. In some embodiments the cancer stem cells are oral squamous carcinoma stem cells. Cancers are optionally profiled prior to treatment to determine the presence of cancer stem cells, where such cells may be identified by the presence of markers known in the art, including without limitation expression of CD44, CD133, etc., where the presence of the cancer stem cells, e.g. at 0.1%, 1%, 2%, 5% or more of the tumor mass is indicative that the individual is suited for treatment by the methods of the invention.

The methods of the invention involve an initial depletion of effector cells in the tumor microenvironment, for example by irradiation, chemotherapy, and the like, in a dose that is sufficient to substantially deplete monocytes present in the tumor microenvironment. NK cells are then delivered to the tumor site, e.g. by localized injection at the site of cancer or in close proximity to the site of cancer, although systemic administration may find use, e.g. when the cancer is metastatic. In some embodiments the NK cells are autologous. In other embodiments the NK cells are allogeneic. Repeated administration of NK cells to lyse cancer stem cells may be required.

After isolation from the host or a donor, where the NK cells are optionally enriched, the NK cells are activated, e.g. with IL-2+1L12 or IL-2+IFN-α, which increases the cytotoxic function of NK cells and expands the numbers of NK cells. Prior to use the NK cells are typically washed free of excess cytokines.

The methods of the invention may be combined with therapy designed to eliminate the differentiated cancer cells present in a tumor, e.g. in a combination therapy with EGFR antibody (Erbitux). The methods of the invention may be combined with additional therapy targeted at cancer stem cells, including, without limitation, a therapeutic dose of a taxane and N-acetylcysteine. Methods of the invention may also be enhanced by blocking or targeted knock-down of COX2 and/or NFκB in the tumor.

Methods for the use of NK cells to establish tolerance to stem cell grafts are also provided. Anti CD16 antibody can be used to tolerize NK cells to support differentiation of stem cells. Anti-CD16 antibody will block cytotoxicity of the NK cells but will induce secretion of IFN-γ which will induce differentiation and resistance of stem cells, thus NK cells will be treated with IL-2 and/or IL-12, or IFN-60 in the presence of anti-CD16 antibody to induce split anergy which will then support differentiation of stem cells. Stem cells will be mixed with monocytes and then injected since monocytes will not only support differentiation and survival of stem cells but also they will induce split anergy in NK cells, making them cells which will contribute to differentiation of stem cells.

Composition and methods are provided for increasing the survival of stem cell and stem cell derived cells during the process of transplantation. Cells of interest for transplantation include differentiated and progenitor cells, which may be derived from tissue sources of progenitor cells or may be derived from the differentiation of suitable stem cells, including embryonic stem cells, induced pluripotent stem cells, etc., where the differentiation is optionally performed in vitro or ex vivo. Various differentiated cells derived from pluripotent cells in vitro include cardiomyocytes, neuronal cells, e.g. neurons, neural progenitors, oligodendrocytes, etc.; pancreatic cells and progenitors thereof, e.g. beta cells, alpha cells, etc.; hematopoietic cells, e.g. hematopoietic stem cells and lineage committed progenitor cells; muscle cells; and the like.

In one embodiment, methods are provided for transplantation, the method comprising administering to a recipient during transplantation of cells derived from stem cells in vitro, stem cells mixed with monocytes and then injected since monocytes will not only support differentiation and survival of stem cells but also they will induce split anergy in NK cells, making them cells which will contribute to differentiation of stem cells. Optionally, the method further comprises detection of viable cells following transplantation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Phenotypic characteristics of UCLA-OSCCs and UCLA-OSCSCs. UCLA-OSCCs or UCLA-OSCSCs were detached , washed and stained with the antibodies recognizing surface receptors indicated in the figure and analyzed by flow cytometry. Isotype control antibodies were used as controls. The numbers on the right hand corner are the mean channel fluorescence intensity. (A). UCLA-OSCCs or UCLA-OSCSCs were left untreated or treated with EGF (10 ng/ml), and the cell extracts were prepared after an overnight incubation, and run on polyacrylamide gel, after which the bands were transferred and blotted with an antibody specifc for phospho-Stat3 (B). UCLA-OSCCs or UCLA-OSCSCs at a density of 2×10⁵ cells per well were transduced with the NFκB-Luciferase lentiviral reporter vector for 48 hours before they were lysed and luciferase activity measured [RLU/s] using a luminometer. An internal lentiviral vector expressing constitutive Luciferase was used for normalization (C). One of three representative experiments is shown in this figure.

FIG. 2. Increased NK cell cytotoxicity against UCLA-OSCSCs. PBMCs and NK cells were left untreated or treated with IL-2 (1000 units/ml) or anti-CD16 mAb (3 μg/ml) or a combination of IL-2 (1000 units/ml) and anti-CD16 mAb (3 μg/ml) for 12-24 hours before they were added to ⁵¹Cr labeled primary oral tumors. PBMC (A) and NK cell (B) cytotoxicity was determined using a standard ⁵¹Cr release assay and the lytic units 30/10⁶ were determined using inverse number of effectors required to lyse 30% of the tumor cells ×100. Differences between untreated, anti-CD16 mAb treated or IL-2 and/or anti-CD16 mAb treated NK cell killing between UCLA-OSCCs and UCLA-OSCSCs were significant at a p value of <0.05. One of four representative experiments is shown in this figure.

FIG. 3. Increased cytotoxicity, decreased secretion of IL-6 and increased secretion of IFN-γ in co-cultures of NK cells with NFκB knock down UCLA-OSCCs and HOK-16B cells. IκB_{(S32AS36A)} transduced UCLA-OSCCs (A) and IκBαM transduced HOK-16B cells (B) and their EGFP transduced controls were transfected with 8 μg of NFκB Luciferase reporter vector and treated with and without TNF-α (20 ng/ml) for 18 hours. The relative Luciferase activity was then determined in the lysates according to the manufacturer's recommendation and fold induction in luciferase activity was determined relative to untreated cells. IκB _{(S32AS36A)} transduced UCLA-OSCCs (C) and IκBαM transduced HOK-16B cells (D) and their EGFP transduced controls were cultured at 2×10⁵ cells/ml, and after an overnight incubation the supernatants were collected and the levels of secreted IL-6 were determined using ELISA specific for IL-6. IκB_{(S32AS36A)} transduced UCLA-OSCCs and IκBαM transduced HOK-16B cells and their EGFP transduced controls were co-cultured with untreated or IL-2 (1000 u/ml) treated NK cells at 1:1 effector to target ratio. After an overnight incubation the supernatants from the co-cultures of UCLA-OSCCs and HOK-16B cells with NK cells were collected and the levels of secreted IL-6 (FIGS. E and F), and IFN-γ (FIGS. G and H) were determined by specific ELISAs for each cytokine. NK cells were left untreated or treated with IL-2 for 12-24 hours before they were added to IκB_{(S32AS36A)} transduced UCLA-OSCCs and IκBαM transduced HOK-16B cells and their EGFP transduced controls. Differences between EGFP transduced and those with either IκB_{(S32AS36A)} transduced UCLA-OSCCs or IκBαM transduced HOK-16B cells were significant for IL-2 treated NK cells at a p value of <0.05. IκB_{(S32AS36A)} transduced UCLA-OSCCs and IκBαM transduced HOK-16B cells and their EGFP transduced controls were ⁵¹Cr labeled before they were co-cultured with untreated or IL-2 (1000 u/ml) treated NK cells. After 4 hours of incubation at 37 C cytotoxicity of NK cells were assessed using a standard ⁵¹Cr release assay (FIGS. I and J). lytic unit 30/10⁶ cells were determined using inverse number of effectors required to lyse 30% of the tumor cells ×100. Differences between IκB_{(S32AS36A)} transduced UCLA-OSCCs or IκBαM transduced HOK-16B cells and those with EGFP transduced were significant in IL-2 treated PBMCs at a p value of <0.05. One of three representative experiments is shown in this figure.

FIG. 4. Lysis of hESCs by untreated and IL-2 treated NK cells is inhibited by anti-CD16 antibody treatment, however, the same treatment induced significant secretion of IFN-γ by the NK cells. NK cells (1×10⁶/ml) were left untreated or treated with IL-2 (1000 units/ml), or anti-CD16 mAb (3 μg/ml) or a combination of IL-2 (1000 units/ml) and anti-CD16 mAb (3 μg/ml) for 12-24 hours before they were added to ⁵¹Cr labeled hESCs. NK cell cytotoxicity was determined using a standard 4 hour ⁵¹Cr release assay, and the lytic units 30/10⁶ were determined using inverse number of NK cells required to lyse 30% of the hESCs ×100 (A). NK cells were treated as described in FIG. 4A and each NK sample at (1×10⁵/ml) were either cultured in the absence of hESCs or added to hESCs at an NK to hESC ratio of 1:1. After an overnight culture, supernatants were removed from the cultures and the levels of IFN-γ (B), and bFGF (C) secretion were determined using specific ELISAs. One of three representative experiments is shown in this figure.

FIG. 5. Lysis of iPS cells by untreated and IL-2 treated NK cells is inhibited by anti-CD16 antibody treatment, however, the same treatment induced significant secretion of IFN-γ by the NK cells. NK cells (1×10⁶/ml) were left untreated or treated with IL-2 (1000 units/ml), or anti-CD16 mAb (3 μg/ml) or a combination of IL-2 (1000 units/ml) and anti-CD16 mAb (3 μg/ml) for 12-24 hours before they were added to ⁵¹Cr labeled iPS cells and NK cell cytotoxicity was determined using a standard 4 hour ⁵¹Cr release assay, and the lytic units 30/10⁶ were determined using inverse number of NK cells required to lyse 30% of the iPS cells ×100 (A). NK cells were treated as described in FIG. 5A and each NK sample at (1×10⁵/ml) were either cultured in the absence of iPS cells or added to iPS cells at an NK to iPS ratio of 1:1. After an overnight culture, supernatants were removed from the cultures and the levels of IFN-γ (B), and bFGF (C) secretion were determined using specific ELISAs. One of two representative experiments is shown in this figure.

FIG. 6. Lysis of DPSCs by untreated and IL-2 treated NK cells is inhibited by anti-CD16 antibody treatment, however, the same treatment induced significant secretion of IFN-γ by the NK cells. NK cells (1×10⁶/ml) were left untreated or treated with IL-2 (1000 units/ml), or anti-CD16 mAb (3 μg/ml) or a combination of IL-2 (1000 units/ml) and anti-CD16 mAb (3 μg/ml) for 12-24 hours before they were added to ⁵¹Cr labeled DPSCs and NK cell cytotoxicity was determined using a standard 4 hour ⁵¹Cr release assay, and the lytic units 30/10⁶ were determined using inverse number of NK cells required to lyse 30% of the DPSCs ×100 (A). NK cells were treated as described in FIG. 6A and each NK sample at (1×10⁵/ml) either cultured in the absence of DPSCs or added to DPSCs at an NK to DPSC ratio of 1:1. After an overnight culture, supernatants were removed from the cultures and the levels of IFN-γ (B), and bFGF (C) secretion were determined using specific ELISAs. One of five representative experiments is shown in this figure.

FIG. 7. Lysis of MSCs by untreated and IL-2 treated NK cells is inhibited by anti-CD16 antibody treatment, however, the same treatment induced significant secretion of IFN-γ by the NK cells. NK cells (1×10⁶/ml) were left untreated or treated with IL-2 (1000 units/ml), or anti-CD16 mAb (3 μg/ml) or a combination of IL-2 (1000 units/ml) and anti-CD16 mAb (3 μg/ml) for 12-24 hours before they were added to ⁵¹Cr labeled MSCs and NK cell cytotoxicity was determined using a standard 4 hour ⁵¹Cr release assay, and the lytic units 30/10⁶ were determined using inverse number of NK cells required to lyse 30% of the MSCs ×100 (A). NK cells were treated as described in FIG. 7A and each NK sample at (1×10⁵/ml) either cultured in the absence of MSCs or added to MSCs at an NK to MSC ratio of 1:1. After an overnight culture, supernatants were removed from the cultures and the levels of IFN-γ (B), and bFGF (C) secretion were determined using specific ELISAs. One of five representative experiments is shown in this figure. Monocytes were purified from PBMCs and irradiated as indicated in the Material and Methods section. MSCs (1×10⁶ cells/plate) were cultured with the irradiated monocytes (monocyte: MSC ratio of 1:1) for 24-48 hours before they were removed from the plates, washed and labeled with ⁵¹Cr and used as targets in the cytotoxicity assays against NK cells. The NK samples were either left untreated or treated with anti-CD16 mAb (3 μg/ml), IL-2 (1000 u/ml), or a combination of IL-2 (1000 u/ml) and anti-CD16 mAb (3 μg/ml) for 24-48 hours before they were added to ⁵¹Cr labeled MSCs at different effector to target (E:T) ratios. Supernatants were removed after 4 hours of incubation and the released radioactivity counted by a γ counter. % cytotoxicity was determined at different E:T ratio, and LU₃₀/10⁶ cells were calculated using the inverse of the number of effectors needed to lyse 30% of the MSCs ×100.. One of three representative experiments is shown in this figure (D). MSCs (1×10⁵ cells/well) were co-cultured with and without irradiated Monocytes at 1:1 MSCs to monocytes for 24-48 hours before untreated or IL-2 (1000 u/ml) pre-treated or anti-CD16 mAb (3 μg/ml) pre-treated, or a combination of IL-2 (1000 u/ml) and anti-CD16 mAb (3 μg/ml) pre-treated NK cells at 1:1:1 NK:monocyte:MSC ratios were added. NK cells were pre-treated as indicated for 24-48 hours before they were added to the co-cultures of monocytes and MSCs. NK samples were also cultured in the absence of monocytes and MSCs. After 24-48 hours of the addition of NK cells the supernatants were removed from the cultures and the levels IFN-γ (E) were determined using ELISA. One of five representative experiments is shown in this figure.

FIG. 8. MSCs are significantly more sensitive to lysis by IL-2 treated NK cells than their differentiated counterparts and they trigger significant release of IFN-γ by IL-2 activated NK cells. MSCs were seeded at 3 to 4×10⁵ cells per well in Stem cell medium in the presence and absence of untreated PBMCs or IL-2 (1000 u/ml) treated PBMCs (PBMC to Stem cell ratio 10:1). After 2 days of co-cultures, Alkaline Phosphatase staining was performed. A1 to C1 (triplicates of MSCs in the absence of PBMCs), A2 to C2 (MSC in the presence of untreated PBMCs), A3 to C3 (MSC in the presence of IL-2 treated PBMCs), A4 (naïve PBMCs alone), B4 (IL-2 treated PBMCs alone) (A). The ALP stain densities for each well were determined using photoshop software (B). MSCs were cultured in differentiation medium for 1 week and differentiated Osteoblasts were then seeded at 3 to 4×10⁵ cells per well in differentiation medium in the presence and absence of untreated PBMCs and IL-2 (1000 u/ml) treated PBMCs (PBMC to Stem cell ratio 10:1). After 2 days of co-cultures Alkaline Phosphatase staining was performed. A1 to C1 (triplicates of Ostoblastic cells in the absence of PBMCs), A2 to C2 (Ostoblastic cells in the presence of untreated PBMCs), A3 to C3 (Ostoblastic cells in the presence of IL-2 treated PBMCs), A4 (untreated PBMCs alone), B4 (IL-2 treated PBMCs alone) (C). The ALP stain densities for each well were determined using photoshop software (D). Stem cells and Osteoblasts were cultured with and without untreated PBMCs as described above and after two days of incubation the supernatants were removed and subjected to specific ELISA for VEGF (E). Undifferentiated MSCs and those differentiated to osteoblasts were cultured in the absence and presence of different concentrations of HEMA as indicated in the figure and the levels of cell death were determined by flow cytometric analysis of PI stained MSCs and osteoblasts after an overnight incubation (F). NK cells (1×10⁶/ml) were left untreated or treated with IL-2 (1000 units/mil), or anti-CD16 mAb (3 μg/ml) or a combination of IL-2 (1000 units/ml) and anti-CD16 mAb (3 μ/ml) for 12-24 hours before they were added to ⁵¹Cr labeled MSCs or osteoblasts, and NK cell cytotoxicity was determined using a standard 4 hour ⁵¹Cr release assay, and the lytic units 30/10⁶ were determined using inverse number of NK cells required to lyse 30% of the MSCs or osteoblasts ×100 (G). Undifferentiated MSCs and those differentiated to osteoblasts at (1×10⁵/ml) were cultured in the absence and presence of untreated NK cells or IL-2 treated NK cells at 1:1 ratio and after two days of incubation the supernatants were removed and subjected to specific ELISA for IFN-γ (H). MSCs at (1×10⁵/ml) were either cultured with untreated NK cells or IL-2 treated NK cells alone (1:1; MSC:NK) or with untreated NK and IL-2 treated NK cells with monocytes at (1:1:1; MSC:NK:monocytes). After an overnight incubation, the cells were washed and B7H1 surface expression was determined on MSC gated populations. Isotype control antibodies were used as controls (I). MSCs were left untreated or treated with IFN-γ (500 u/ml). After an overnight incubation, MSCs were washed and the B7H1 surface expression was determined on MSC.

FIG. 9. Undifferentiated DPSCs are significantly more sensitive to lysis by IL-2 treated NK cells than their differentiated counterparts. NK cells (1×10⁶/ml) were left untreated or treated with IL-2 (1000 units/ml), or anti-CD16 mAb (3 μg/ml) or a combination of IL-2 (1000 units/ml) and anti-CD16 mAb (3 μg/ml) for 12-24 hours before they were added to ⁵¹Cr labeled undifferentiated and differentiated DPSCs and NK cell cytotoxicity was determined using a standard 4 hour ⁵¹Cr release assay. Lytic units 30/10⁶ were determined using inverse number of NK cells required to lyse 30% of the DPSCs ×100. Passage 8 differentiated and undifferentiated DPSCs were used.

FIG. 10. Monocytes are significantly more sensitive to NK cell mediated cytotoxicity than DCs. NK cells (1×10⁶/ml) were left untreated or treated with IL-2 (1000 units/ml), or anti-CD16 mAb (3 μg/ml) or a combination of IL-2 (1000 units/ml) and anti-CD16 mAb (3 μg/ml) for 12-24 hours before they were added to ⁵¹Cr labeled autologous monocytes or ⁵¹Cr labeled autologous DCs, and NK cell cytotoxicity were determined using a standard 4 hour ⁵¹Cr release assay and the lytic units 30/10⁶ were determined using inverse number of NK cells required to lyse 30% of the monocytes or DCs ×100. One of four representative experiments is shown in this figure.

FIG. 11. IPS cells are more susceptible to NK cell mediated cytotoxicity than their parental line. NK cells (1×10⁶/ml) were left untreated or treated with IL-2 (1000 units/ml), or anti-CD16 mAb (3 μg/ml) or a combination of IL-2 (1000 units/ml) and anti-CD16 mAb (3 μg/ml) for 12-24 hours before they were added to ⁵¹Cr labeled iPS cells or ⁵¹Cr labeled parental cells from which the iPS cells were derived, and NK cell cytotoxicity were determined using a standard 4 hour ⁵¹Cr release assay and the lytic units 30/10⁶ were determined using inverse number of NK cells required to lyse 30% of the iPS or parental cells ×100.

FIG. 12. Targeted inhibition of COX2 in bone marrow monocytes increased NK cell cytotoxicity and secretion of IFN-γ by IL-2 treated NK cells. Purified NK cells and monocytes were obtained from spleens and bone marrows of 3 pooled control mice and those with targeted knock down of COX2 gene in myeloid cells respectively (n=3). Purified NK cells and monocytes from control mice and those with targeted knock down of COX2 gene in myeloid cells were then cultured with and without IL-2 (1000 u/ml) at 1:1 NK: monocyte ratios for 6 days before they were added to ⁵¹Cr labeled YAC cells, and NK cell cytotoxicity was determined in 4 hours ⁵¹Cr release assay. The lytic units 30/10⁶ were determined using inverse number of NK cells required to lyse 30% of the YAC cells ×100 (A). NK cells were cultured as described in FIG. 11A and after 6 days of incubation the supernatants were removed and IFN-γ secretion were measured in the supernatants using a specific ELISA (B). One of five representative experiments is shown in this figure.

FIG. 13. Schematic representation of hypothetical model of oral cancer stem cell differentiation by NK cells and monocytes. Interaction of cancer stem cells or primary stem cells with monocytes and NK cells results in the loss of NK cell cytotoxicity due partly to the induction of resistance of cancer stem cells by monocytes and indirectly by monocytes serving as targets of NK cells, thus serving as a shield which protects the stem cells from lysis by the NK cells. Loss of NK cell cytotoxicity by monocytes and gain in secretion of IFN-γ results in a significant induction of transcription factors, cytokines and growth factors in stem cells and differentiation of stem cells.

FIG. 14. Treatment of PBMCs or NK cells with anti-CD16 mAb decreased cytotoxicity significantly against both tumor types.

FIG. 15. Resistance of Cancer stem cells to HEMA and cisplatinum mediated cell death.

FIG. 16. Resistance of Cancer stem cells to radiation and cisplatinum treatment.

FIG. 17. Lysis of cancer stem cells by the combination of NAC and Paclitaxel and Paclitaxel alone.

FIGS. 18A-D. Lysis of stem cells by NK cells.

FIGS. 19A-19D. Stem cells triggered significant secretion of IFN-γ from IL-2 treated NK cells when compared to IL-2 treated NK cells in the absence of stem cells.

FIGS. 20A-20B. IL-2 treated NK cells secreted moderate amounts of IFN-γ which were synergistically increased when co-cultured in the presence of MSCs.

FIGS. 21A-21C. Differentiated DPSCs are more resistant to NK cell mediated cytotoxicity.

FIGS. 22A-B. Increased NK cell cytotoxicity against UCLA-OSCSCs but not those of UCLA-OSCCs.

FIGS. 23A-F. Blocking NFκB in UCLA-OSCCs and HOK-16B oral epithelial cells lowered IL-6 to IFN-γ ratios and increased their sensitivity to NK cell mediated cytotoxicity.

FIGS. 24A-24B. Bioluminescent tracking of engrafted HESC in RAG2-/-gc-/- Mice.

FIG. 25. Dose dependent effect of cisplatin and Paclitaxel on two oral tumors.

FIG. 26. Dose dependent increase in cisplatin mediated killing of pancreatic cells.

FIG. 27. BxPC3 is less differentiated, and a more stem-like pancreatic tumor line based on surface analysis, and more sensitive to NK cell mediated cytotoxicity.

FIG. 28A-28B. Dose dependent synergistic induction of cell death by NAC and Paclitaxel in BXPC3 pancreatic cells,.

FIG. 29. The cytotoxic function of purified NK cells were assessed against lung tumors (A549), Breast tumors (MCF7) and Prostate tumors (PC3).

FIG. 30. Dose dependent synergistic effect of NAC and Paclitaxel on lung (A549), prostate (PC3) and breast (MCF7) tumors.

FIG. 31. A549 lung tumors were cultured with supernatants removed from untreated NK, anti-CD16 mAb treated NKs, IL-2 treated NKs and IL2 in combination of anti-CD16 mAb treated NK cells in the presence and absence of monocytes.

FIG. 32. A549 lung tumors were cultured with supernatants removed from untreated NK, anti-CD16 mAb treated NKs, IL-2 treated NKs and IL2 in combination of anti-CD16 mAb treated NK cells in the presence and absence of monocytes.

### DETAILED DESCRIPTION OF THE INVENTION

Compositions and methods are provided for the treatment of cancer with NK cells, and for transplantation of stem cells, including pluripotent stem cells, e.g. iPS cells, embryonic stem cells, etc. and for the transplantation of differentiated cells derived from such stem cells, usually derived from such stem cells in vitro.

The methods of the invention involve an initial depletion of effector cells in the tumor microenvironment, for example by irradiation, chemotherapy, and the like, in a dose that is sufficient to substantially deplete monocytes present in the tumor microenvironment. NK cells are then delivered to the tumor site. In some embodiments the NK cells are autologous. In other embodiments the NK cells are allogeneic. Repeated administration of NK cells to lyse cancer stem cells may be required.

The methods of the invention may be combined with therapy designed to eliminate the differentiated cancer cells present in a tumor, e.g. in a combination therapy with EGFR antibody (Erbitux). The methods of the invention may be combined with additional therapy targeted at cancer stem cells, including, without limitation, a therapeutic dose of a taxane and N-acetylcysteine methods of the invention may also be enhanced by blocking or targeted knock-down of COX2 and/or NFκB in the tumor.

As used herein, a recipient is an individual to whom tissue or cells from another individual (donor), commonly of the same species, has been transferred. Generally the MHC antigens, which may be Class I or Class II, will be matched, although one or more of the MHC antigens may be different in the donor as compared to the recipient. The recipient and donor are generally mammals, preferably human. Laboratory animals, such as rodents, e.g. mice, rats, etc. are of interest for drug screening, elucidation of developmental pathways, etc. For the purposes of the invention, the cells may be allogeneic, autologous, or xenogeneic with respect to the recipient.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

Cancer stem cells. It is well documented that many types of tumors contain cancer cells with heterogeneous phenotypes, reflecting aspects of the differentiation that normally occurs in the tissues from which the tumors arise. The variable expression of normal differentiation markers by cancer cells in a tumor suggests that some of the heterogeneity in tumors arises as a result of the anomalous differentiation of tumor cells. It has been shown for solid cancers that the cells are phenotypically heterogeneous and that only a small proportion of cells are clonogenic in culture and in vivo. Tumorigenic and non-tumorigenic populations of cancer cells can be isolated based on their expression of cell surface markers. In many cases of breast cancer, only a small subpopulation of cells had the ability to form new tumors.

The presence of cancer stem cells has profound implications for cancer therapy. For many years, however, it has been recognized that small numbers of disseminated cancer cells can be detected at sites distant from primary tumors in patients that never manifest metastatic disease. Most cancer cells lack the ability to form a new tumor such, that only the dissemination of rare cancer stem cells can lead to metastatic disease. If so, the goal of therapy must be to identify and kill this cancer stem cell population. Squamous carcinoma stem cells (SCSC) are known in the art to be positive for expression of CD44 and CD133, and negative for expression of specific lineage markers.

Samples, including tissue sections, slides, etc. containing a squamous carcinoma tissue, are optionally stained with reagents specific for markers that indicate the presence of cancer stem cells. Samples may be frozen, embedded, present in a tissue microarray, and the like. The reagents, e.g. antibodies, polynucleotide probes, etc. may be detectably labeled, or may be indirectly labeled in the staining procedure. The data provided herein demonstrate that the number and distribution of progenitor cells is diagnostic of the stage of the carcinoma.

The invention finds use in the treatment of squamous cell carcinomas. Carcinomas are malignancies that originate in the epithelial tissues. Epithelial cells cover the external surface of the body, line the internal cavities, and form the lining of glandular tissues. In adults, carcinomas are the most common forms of cancer.

“Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and use of therametrics (e.g., monitoring a subject's condition to provide information as to the effect or efficacy of therapy).

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans. Other subjects may include cattle, dogs, cats, guinea pigs, rabbits, rats, mice, horses, and the like.

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

The term “normal” as used in the context of “normal cell,” is meant to refer to a cell of an untransformed phenotype or exhibiting a morphology of a non-transformed cell of the tissue type being examined.

“Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

“Therapeutic target” generally refers to a gene or gene product that, upon modulation of its activity (e.g., by modulation of expression, biological activity, and the like), can provide for modulation of the cancerous phenotype.

Squamous cells are flat cells which form the surface of an epithelium. They can be identified histologically by the fact that they look flattened and thin under a microscope. Epithelia lined by squamous cells can be classified as either simple squamous epithelium or stratified squamous epithelium.

Squamous cell carcinoma is a carcinoma that may occur in many different organs, including the skin, mouth, esophagus, lungs, and cervix. It is a malignant tumor of epithelium that shows squamous cell differentiation. Squamous cell carcinoma is usually developed in the epithelial layer of the skin and sometimes in various mucous membranes of the body. This type of cancer can be seen on the skin, lips, inside the mouth, throat or esophagus.

The most common noncutaneous tumor of the head and neck is squamous cell carcinoma of the larynx, followed by squamous cell carcinomas of the palatine tonsil, tongue, and floor of the mouth. Somewhat less common are tumors of the salivary gland, jaw, nose and paranasal sinuses, and ear. Tumors of the thyroid gland, eye, and skin are discussed elsewhere in the manual. Excluding the skin and thyroid gland, >90% of head and neck cancers are squamous cell (epidermoid) carcinomas, and 5% are melanomas, lymphomas, and sarcomas. The Epstein-Barr virus plays a role in the pathogenesis of nasopharyngeal cancer.

Oral squamous cell carcinoma affects about 30,000 Americans each year. Oral squamous cell carcinoma is the most common oral or pharyngeal cancer. The chief risk factors for oral squamous cell carcinoma are smoking and alcohol use. Squamous cell carcinoma of the tongue may also result from Plummer-Vinson syndrome, syphilis, or chronic trauma. About 40% of intraoral squamous cell carcinomas begin on the floor of the mouth or on the lateral and ventral surfaces of the tongue. About 38% of all oral squamous cell carcinomas occur on the lower lip, and about 11% begin in the palate and tonsillar area.

If carcinoma of the tongue is localized (no lymph node involvement), 5-yr survival is about 50%. For localized carcinoma of the floor of the mouth, 5-yr survival is 65%. With lymph node metastasis, the 5-yr survival is 20%. For lower lip lesions, 5-yr survival is 90%, and metastases are rare. Carcinoma of the upper lip tends to be more aggressive and metastatic. For carcinoma of the palate and tonsillar area, 5-yr survival is 68% if patients are treated before lymph node involvement but only 17% after involvement. Metastases reach the regional lymph nodes first and later the lungs. Surgery and radiation therapy are the treatments of choice.

About 90% of vulvar cancers are squamous cell carcinomas. Vulvar cancer most often occurs in elderly women. It usually manifests as a palpable lesion. Diagnosis is by biopsy. Treatment includes excision and inguinal and femoral lymph node dissection. Vulvar cancer accounts for about 3 to 4% of gynecologic cancers in the US. Average age at diagnosis is about 70, and incidence increases with age. Risk factors include vulvar intraepithelial neoplasia (VIN), human papillomavirus infection, heavy cigarette smoking, lichen sclerosus, squamous hyperplasia, squamous carcinoma of vagina or cervix, and chronic granulomatous diseases. VIN is a precursor to vulvar cancer. VIN may be multifocal. Sometimes adenocarcinoma of the vulva, breast, or Bartholin's glands also develops.

Squamous cell carcinoma of the skin is a malignant tumor of epidermal keratinocytes that invades the dermis, usually occurring in sun-exposed areas. The incidence in the US is 80,000 to 100,000 cases annually, with 2000 deaths. Local destruction may be extensive, and metastases occur in advanced stages. Diagnosis is by biopsy. Treatment depends on the tumor's characteristics and may involve curettage and electrodesiccation, surgical excision, cryosurgery, or, occasionally, radiation therapy.

Squamous cell carcinoma is the most common malignancy of the larynx. In the US, it is 4 times more common in men and is more common among blacks than whites. Over 95% of patients are smokers; 15 pack-years of smoking increases the risk 30-fold. Sixty percent of patients present with localized disease alone, 25% with local disease and regional nodal metastatic disease, and 15% with advanced disease, distant metastases, or both. Common sites of origin are the true vocal cords (glottis) particularly the anterior portion, supraglottic larynx (epiglottis), hypopharynx (pyriform sinus), and postcricoid area.

The most common malignant esophageal tumor is squamous cell carcinoma. Symptoms are progressive dysphagia and weight loss. Diagnosis is by endoscopy, followed by CT and endoscopic ultrasound for staging. Treatment varies with stage and generally includes surgery with or without chemotherapy and radiation. Long-term survival is poor except for those with local disease. About 8000 cases of esophageal squamous cell carcinoma occur annually in the US.

About 80 to 85% of all cervical cancers are squamous cell carcinoma. Diagnosis is by screening cervical Papanicolaou (Pap) test and biopsy. Staging is clinical. Treatment usually includes surgical resection, radiation therapy, and, unless cancer is localized, chemotherapy; if cancer is widely metastasized, treatment is primarily chemotherapy. Cervical cancer results from cervical intraepithelial neoplasia (CIN), which appears to be caused by infection with human papillomavirus (HPV) type 16, 18, 31, 33, 35, or 39.

CIN is graded as 1 (mild cervical dysplasia), 2 (moderate dysplasia), or 3 (severe dysplasia and carcinoma in situ). CIN 3 is unlikely to regress spontaneously; if untreated, it may, over months or years, penetrate the basement membrane, becoming invasive carcinoma. Invasive cervical cancer usually spreads by direct extension into surrounding tissues or via the lymphatics to the pelvic and para-aortic lymph nodes. Hematogenous spread is possible.

Natural killer cells (or NK cells) are a type of cytotoxic lymphocyte that constitute a major component of the innate immune system. NK cells play a major role in the rejection of tumors and cells infected by viruses. They usually express the surface markers CD16 (FcγRIII) and CD56 in humans. Given their strong cytolytic activity and the potential for auto-reactivity, NK cell activity is tightly regulated. NK cells must receive an activating signal, which can come in a variety of forms. NK cells are activated in response to interferons or macrophage-derived cytokines. Cytokines involved in NK activation include IL-12, IL-15, IL-18, IL-2, and CCL5.

NK cells may be isolated by negative or positive selection using methods and reagents known in the art. For example negative selection may utilize commercially available antibodies that bind to CD3, CD4, CD19, CD66b, glycophorin, etc. Alternatively NK cells may be positively selected for expression of CD56, and/or CD16.

Taxanes are a class of chemotherapeutic agent, which include, without limitation, the following compounds:

Taxane List

| Common | Trade | |
|---|---|---|
| Name | Name | Structure |
| paclitaxel | Taxol | 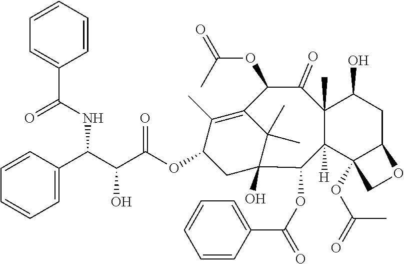 |
| docetaxel | Taxotere | 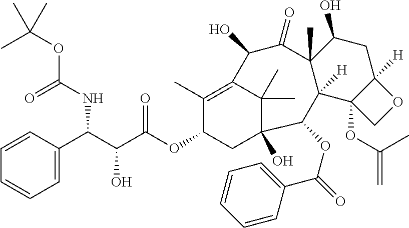 |
| MAC 32 TL 139 | Milataxel | 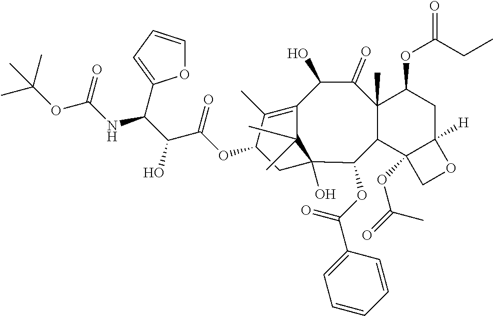 |
| TL-909; MST- 997 | Simotaxel | 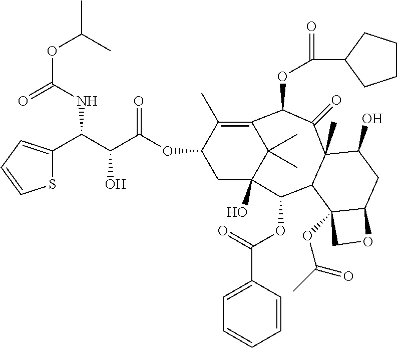 |
| TL-310 | | 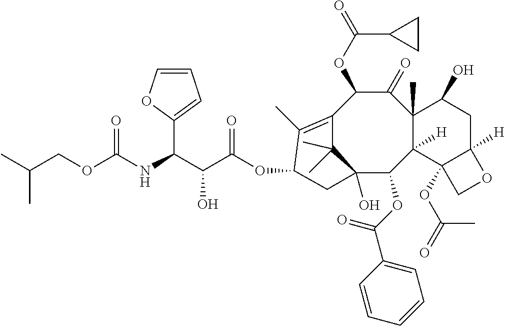 |
| BMS-184476 | | 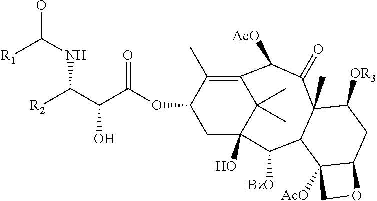 |
| BMS-275183 | | 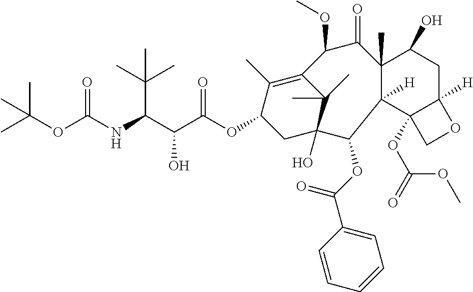 |
| DJ-927 | Tesetaxel | 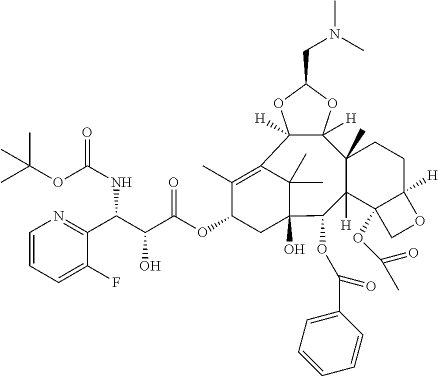 |
| RPR 109881; RPR 109881A | Larotaxel | 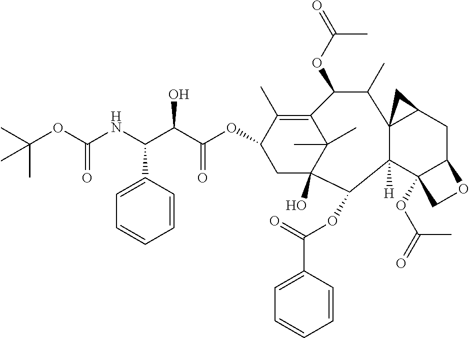 |
| Bay-59-8862, IDN-5109, SB-T- 101131 | Ortataxel | 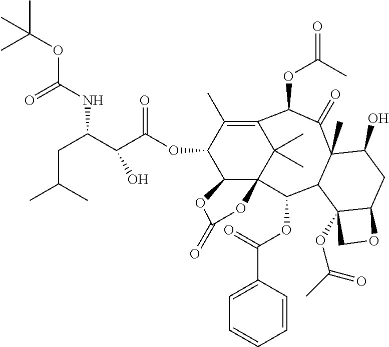 |
| Docosahexaenoyl (DHA)-paclitaxel | Taxoprexin | 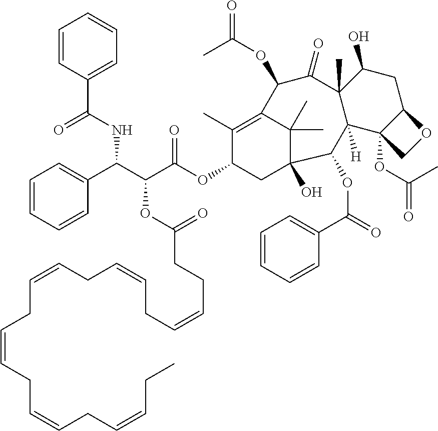 |
| TPI-287 | | 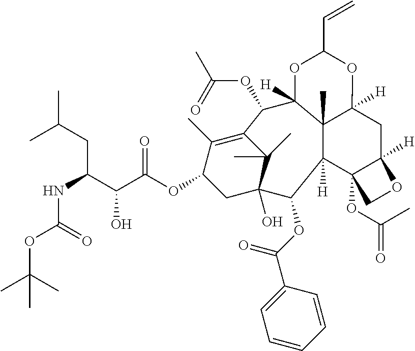 |
| CT-2103 (paclitaxel poliglumex) | Xyotax Opaxio | 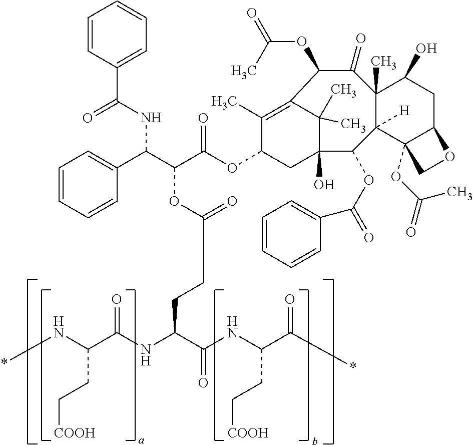 |

As used throughout, “modulation” is meant to refer to an increase or a decrease in the indicated phenomenon (e.g., modulation of a biological activity refers to an increase in a biological activity or a decrease in a biological activity).

By “pluripotency” and pluripotent stem cells it is meant that such cells have the ability to differentiate into all types of cells in an adult organism. The term “induced pluripotent stem cell” encompasses pluripotent cells, that, like embryonic stem (ES) cells, can be cultured over a long period of time while maintaining the ability to differentiate into all types of cells in an organism, but that, unlike ES cells (which are derived from the inner cell mass of blastocysts), are derived from differentiated somatic cells, that is, cells that had a narrower, more defined potential and that in the absence of experimental manipulation could not give rise to all types of cells in the organism. By “having the potential to become iPS cells” it is meant that the differentiated somatic cells can be induced to become, i.e. can be reprogrammed to become, iPS cells. In other words, the somatic cell can be induced to redifferentiate so as to establish cells having the morphological characteristics, growth ability and pluripotency of pluripotent cells. iPS cells have an hESC-like morphology, growing as flat colonies with large nucleo-cytoplasmic ratios, defined borders and prominent nucleoli. In addition, iPS cells express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, pluripotent cells are capable of forming teratomas. In addition, they are capable of forming or contributing to ectoderm, mesoderm, or endoderm tissues in a living organism.

Stem cells and cultures thereof. Pluripotent stem cells are cells derived from any kind of tissue (usually embryonic tissue such as fetal or pre-fetal tissue), which stem cells have the characteristic of being capable under appropriate conditions of producing progeny of different cell types that are derivatives of all of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)).

Stem cells of interest also include embryonic cells of various types, exemplified by human iPS and human embryonic stem (hES) cells, described by Thomson et al: (1998) Science 282:1145; embryonic stem cells from other primates, such as Rhesus stem cells (Thomson et al. (1995) Proc. Natl. Acad. Sci USA 92:7844); marmoset stem cells (Thomson et a/. (1996) Biol. Reprod. 55:254); and human embryonic germ (hEG) cells (Shamblott et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection.

Progenitor or Differentiated Cells. A “differentiated cell” is a cell that has progressed further down the developmental pathway than the cell it is being compared with. Thus, embryonic stem cells can differentiate to lineage-restricted progenitor cells (such as a mesodermal stem cell), which in turn can differentiate into other types of progenitor cells further down the pathway (such as an cardiomyocyte progenitor), and then to an end-stage differentiated cell, which plays a characteristic role in a certain tissue type, and may or may not retain the capacity to proliferate, further. For the purposes of the present invention, progenitor cells are those cells that are committed to a lineage of interest, but have not yet differentiated into a mature cell.

The potential of ES cells to give rise to all differentiated cells provides a means of giving rose to any mammalian cell type, and so a very wide range of culture conditions may be used to induce differentiation, and a wide range of markers may be used for selection. One of skill in the art will be able to select markers appropriate for the desired cell type.

Among the differentiated cells of interest are cells not readily grown from somatic stem cells, or cells that may be required in large numbers and hence are not readily produced in useful quantities by somatic stem cells. Such cells may include, without limitation, neural cells, oligodendrocytes, pancreatic islet cells, hematopoietic cells, cardiac muscle cells, etc.

For example, NCAM may be used as a marker for the selection of aggregates comprising neural lineage cells, inter alia (see Kawasaki et al. (2002) PNAS 99:1580-1585). Neuronal subpopulations can be derived from in vitro differentiation of embryonic stem (ES) cells by treatment of embryo-like aggregates with retinoic acid (RA). The cells express Pax-6, a protein expressed by ventral central nervous system (CNS) progenitors. CNS neuronal subpopulations generated expressed combinations of markers characteristic of somatic motoneurons (Islet-1/2, Lim-3, and HB-9), cranial motoneurons (Islet-1/2 and Phox2b) and interneurons (Lim-1/2 or EN1) (Renoncourt et al. (1998) Mech Dev. 179(1-2):185-97; Harper et al. (2004) PNAS 101(18):7123-8).

Another lineage of interest is pancreatic cells. The pancreas is composed of exocrine and endocrine compartments. The endocrine compartment consists of islets of Langerhans, clusters of four cell types that synthesize peptide hormones: insulin (β cells), glucagon (α cells), somatostatin (γ cells), and pancreatic polypeptide (PP cells). Although the adult pancreas and central nervous system (CNS) have distinct origins and functions, similar mechanisms control the development of both organs. Strategies that induce production of neural cells from ES cells can be adapted for endocrine pancreatic cells. Useful culture conditions include plating EBs into a serum-free medium, expansion in the presence of basic fibroblast growth factor (bFGF), followed by mitogen withdrawal to promote cessation of cell division and differentiation.

Expression of nestin may be useful as a marker for selection of a number of progenitor cells from embryoid bodies. The cells in the pancreatic lineages express GATA-4 and HNF3, as well as markers of pancreatic γ cell fate, including the insulin I, insulin II, islet amyloid polypeptide (IAPP), and the glucose transporter-2 (GLUT 2). Glucagon, a marker for the pancreatic α cell, may also induced in differentiated cells. The pancreatic transcription factor PDX-1 is expressed. These ES cell-derived differentiating cells have been shown to self-assemble into structures resembling pancreatic islets both topologically and functionally (Lumeisky et al. (2001) Science 292(5520):1389-94.

Derivation of hematopoietic lineage cells is also of interest. Hematopoietic stem cells and precursors have been well-characterized, and markers for the selection thereof are well known in the art, e.g. CD34, CD90, c-kit, etc. Co-culture of human ES cells with irradiated bone marrow stromal cell lines in the presence of fetal bovine serum (FBS), but without other exogenous cytokines, leads to differentiation of the human ES cells within a matter of days. A portion of these differentiated cells express CD34, the best-defined marker for early hematopoietic cells (Kaufman and Thomson (2002) J Anat. 200(Pt 3):243-8). CD34⁺ and CD34⁺CD38⁻ cells derived from ES cell cultures have a high degree of similarity in the expression of genes associated with hematopoietic differentiation, homing, and engraftment with fresh or cultured bone marrow (Lu et al. (2002) Stem Cells 20(5):428-37.

A “cardiomyocyte precursor” is defined as a cell that is capable (without dedifferentiation or reprogramming) of giving rise to progeny that include cardiomyocytes. Such precursors may express markers typical of the lineage, including, without limitation, cardiac troponin I (cTnl), cardiac troponin T (cTnT), sarcomeric myosin heavy chain (MHC), GATA-4, Nkx2.5, N-cadherin, β1-adrenoceptor (β1-AR), ANF, the MEF-2 family of transcription factors, creatine kinase MB (CK-MB), myoglobin, or atrial natriuretic factor (ANF).

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

### Methods of Treatment

Compositions and methods are provided for treatment of cancer, particularly the killing of cancer stem cells, by administration of a composition of natural killer cells. In some embodiments the cancer stem cells are carcinoma stem cells, including without limitation squamous carcinoma stem cells. In some embodiments the cancer stem cells are oral squamous carcinoma stem cells. Cancers are optionally profiled prior to treatment to determine the presence of cancer stem cells, where such cells may be identified by the presence of markers known in the art, including without limitation expression of CD44, CD133, etc., where the presence of the cancer stem cells, e.g. at 0.1%, 1%, 2%, 5% or more of the tumor mass is indicative that the individual is suited for treatment by the methods of the invention.

The methods of the invention involve an initial depletion of effector cells in the tumor microenvironment, for example by irradiation, chemotherapy, and the like, in a dose that is sufficient to substantially deplete monocytes present in the tumor microenvironment. The dose appropriate for the individual may be determined based on the evaluation of the patient, the drug or radiation therapy that is selected, the size and phenotype of the tumor mass, and the like.

NK cells are then delivered to the tumor site, e.g. by localized injection at the site of cancer or in close proximity to the site of cancer, although systemic administration may find use, e.g. when the cancer is metastatic. In some embodiments the NK cells are autologous. In other embodiments the NK cells are allogeneic. Repeated administration of NK cells to lyse cancer stem cells may be required. The effective dose of NK cells may be at least about 10⁵ cells, at least about 10⁶ cells, at least about 10⁷ cells, or more.

Generally the NK cells for use in the methods of the invention have been selected, e.g. by positive or negative selection, from an appropriate cell source, e.g. peripheral blood monocytes (PBMC), etc. Methods and markers for the enrichment of NK cells are known in the art. For example negative selection may utilize commercially available antibodies that bind to CD3, CD4, CD19, CD66b, glycophorin, etc. Alternatively NK cells may be positively selected for expression of CD56, and/or CD16. After isolation from the hok or a donor, the NK cells are activated, e.g. with an effective dose of IL-2, e.g. at least about 10 units/ml, at least about 100 units/ml, at least about 1000 units/ml or more, which increases the cytotoxic function of NK cells and expands the numbers of NK cells. The IL-2 is optionally combined with one or more of IL12 and IFN-α at a dose effective to enhance cytotoxicity and expansion of the NK cells. Cells are cultured in the cytokines for a period of time sufficient for activation, e.g. at least about 12 hours, at least about 24 hours, at least about 48 hours and not more than about 4 days, usually not more than about 3 days. Prior to use the NK cells may be typically washed free of excess cytokines. The cells are typically resuspended in an pharmaceutically acceptable excipient, and injected into the patient at an intra-tumoral or systemic site, e.g. i.v., sub-cutaneous, intramuscular, etc.

The methods of the invention may be combined with therapy designed to eliminate the differentiated cancer cells present in a tumor, e.g. in a combination therapy with EGFR antibody (Erbitux), or various other conventional methods of treating differentiated cancer cells.

The methods of the invention may be combined with additional therapy targeted at cancer stem cells, including, without limitation, a therapeutic dose of a taxane and N-acetylcysteine. Alternative, the combination of a taxane and NAC may be used in the absence of NK cells. Taxanes of interest are described herein and include without limitation paclitaxel. The taxane may be provided at a dose that is conventional for the selected agent or at a dose that is less than a conventional dose, e.g. paclitaxel may be used at a dose of from about 25 mg/m², 50 mg/m²; 75 mg/m²; 100 mg/m²; 125 mg/m²; 150 mg/m²; 175 mg/m²; 200 mg/m²; 225 mg/m²; and in combination with N-acetylcysteine, which is shown herein to synergize with taxanes to kill cancer stem cells. N-acetylcysteine may be administered in, for example, an oral dose, at a dose of from about 10 mg/kg body weight; 25 mg/kg body weight; 50 mg/kg body weight; 75 mg/kg body weight; 100 mg/kg body weight; 125 mg/kg body weight; 150 mg/kg body weight; 175 mg/kg body weight; 200 mg/kg body weight. Dosing may be repeated twice or more daily, daily, semi-weekly, weekly, and will generally be monitored for reduction of tumor cells.

An effective amount of such agents can readily be determined by, routine experimentation, as can the most effective and convenient route of administration and the most appropriate formulation. Various formulations and drug delivery systems are available in the art. (See, e.g., Remington's Pharmaceutical Sciences, supra.)

Suitable routes of administration may, for example, include oral, rectal, transmucosal, transdermal, nasal, or intestinal administration and parenteral delivery, including intramuscular, subcutaneous, intramedullary injections, as well as intrathecal, direct intraventricular, intravenous, intraperitoneal, intranasal, or intraocular injections. The agent or composition thereof may be administered in a local rather than a systemic manner. For example, a suitable agent can be delivered via injection or in a targeted drug delivery system, such as a depot or sustained release formulation.

The pharmaceutical compositions may be manufactured by any of the methods well-known in the art, such as by conventional mixing, dissolving, granulating, dragee-making, levigating, emulsifying, encapsulating, entrapping, or lyophilizing processes. The compositions can include one or more physiologically acceptable carriers such as excipients and auxiliaries that facilitate processing of active molecules into preparations for pharmaceutical use. Proper formulation is dependent upon the route of administration chosen.

For example, for injection, the composition may be formulated in aqueous solutions, preferably in physiologically compatible buffers such as Hanks's solution, Ringer's solution, or physiological saline buffer. For transmucosal or nasal administration, penetrants appropriate to the barrier to be permeated are used in the formulation. Such penetrants are generally known in the art. For oral administration, the agents can be formulated readily by combining the active agents with pharmaceutically acceptable carriers well known in the art. Such carriers enable the agents of the invention to be formulated as tablets, pills, dragees, capsules, liquids, gels, syrups, slurries, suspensions and the like, for oral ingestion by a subject. The agents may also be formulated in rectal compositions such as suppositories or retention enemas, e.g., containing conventional suppository bases such as cocoa butter or other glycerides.

Pharmaceutical preparations for oral use can be obtained as solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Pharmaceutical preparations for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration.

Compositions formulated for parenteral administration by injection, e.g., by bolus injection or continuous infusion can be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Formulations for parenteral administration include aqueous solutions of the compound or agent to be administered, including in water-soluble form.

Suspensions of the active agents may also be prepared as appropriate oily injection suspensions. Suitable lipophilic solvents or vehicles include fatty oils such as sesame oil and synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the agents to allow for the preparation of highly concentrated solutions. Alternatively, the active ingredient may be in powder form for constitution with a suitable vehicle, e.g., sterile pyrogen-free water, before use.

For any composition employed herein, a therapeutically effective dose can be estimated initially using a variety of techniques well-known in the art. For example, in a cell culture assay, a dose can be formulated in animal models to achieve a circulating concentration range that includes the IC₅₀ as determined in cell culture. Dosage ranges appropriate for human subjects can be determined, using data obtained from cell culture assays and other animal studies.

A therapeutically effective dose of an agent refers to that amount of the agent that results in amelioration of symptoms or a prolongation of survival in a subject. Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is the therapeutic index, which can be expressed as the ratio LD₅₀/ED₅₀. Agents that exhibit high therapeutic indices are preferred.

Dosages preferably fall within a range of circulating concentrations that includes the ED₅₀ with little or no toxicity. Dosages may vary within this range depending upon the dosage form employed and the route of administration utilized. The exact formulation, route of administration, and dosage should be chosen, according to methods known in the art, in view of the specifics of a subject's condition.

The MEC will vary for each agent but can be estimated from, for example, in vitro data, such as the concentration necessary to achieve 50-90% inhibition of activity using the assays described herein. Dosages necessary to achieve the MEC will depend on individual characteristics and route of administration. Agents or compositions thereof should be administered using a regimen which maintains plasma levels above the MEC for about 10-90% of the duration of treatment, preferably about 30-90% of the duration of treatment, and most preferably between 50-90%. In cases of local administration or selective uptake, the effective local concentration of the drug may not be related to plasma concentration.

The amount of agent or composition administered will, of course, be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack. The pack or dispenser device may be accompanied by instructions for administration. Compositions comprising a agent of the invention formulated in a compatible pharmaceutical carrier may also be prepared, placed in an appropriate container, and labeled for treatment of an indicated condition. Suitable conditions indicated on the label may include treatment of disorders or diseases, such as squamous cell carcinoma or other cancers and conditions associated with altered expression of laminin 332 γ2 domain IV and/or domain V peptides.

### Methods of Transplantation

Ex vivo and in vitro stem cell populations useful as a source of cells may be obtained from any mammalian species, e.g. human, primate, equine, bovine, porcine, canine, feline, etc., particularly human cells. Ex vivo and in vitro differentiated cell populations may include fresh or frozen cells, which may be from a neonate, a juvenile or an adult, and differentiated tissues including skin, muscle, blood, liver, pancreas, lung, intestine, stomach, and other differentiated tissues. Pluripotent cells are optionally deleted from the differentiated cell population prior to introduction into the recipient. The dose of cells will be determined based on the specific nature of the cell, recipient and nature of condition to be treated, and will generally include from about 10⁶-10¹⁰ cells, which may be provided in suspension, as aggregates, and the like.

To determine the suitability of cell compositions for therapeutic administration, the cells can first be tested in a suitable animal model. At one level, cells are assessed for their ability to survive and maintain their phenotype in vivo. The presence and phenotype of the administered cells can be assessed by immunohistochemistry or ELISA using human-specific antibody, or by RT-PCR analysis using primers and hybridization conditions that cause amplification to be specific for human polynucleotides, according to published sequence data.

The stem cells may be used for tissue reconstitution or regeneration in a human patient or other subject in need of such treatment. The cells are administered in a manner that permits them to graft or migrate to the intended tissue site and reconstitute or regenerate the functionally deficient area.

The stem cells may be administered in any physiologically acceptable excipient, where the cells may find an appropriate site for regeneration and differentiation. The cells may be introduced by injection, catheter, or the like. The cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or feeder cells associated with progenitor cell proliferation and differentiation.

The cells of this invention can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types, especially endothelial cells.

Cells may be genetically altered in order to introduce genes useful in the differentiated cell, e.g. repair of a genetic defect in an individual, selectable marker, etc., or genes useful in selection against undifferentiated ES cells. Cells may also be genetically modified to enhance survival, control proliferation, and the like. Cells may be genetically altering by transfection or transduction with a suitable vector, homologous recombination, or other appropriate technique, so that they express a gene of interest. In one embodiment, cells are transfected with genes encoding a telomerase catalytic component (TERT), typically under a heterologous promoter that increases telomerase expression beyond what occurs under the endogenous promoter, (see International Patent Application WO 98/14592). In other embodiments, a selectable marker is introduced, to provide for greater purity of the desired differentiating cell. Cells may be genetically altered using vector containing supernatants over a 8-16 h period, and then exchanged into growth medium for 1-2 days. Genetically altered cells are selected using a drug selection agent such as puromycin, G418, or blasticidin, and then recultured.

The invention now being fully described, it will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

For further elaboration of general techniques useful in the practice of this invention, the practitioner can refer to standard textbooks and reviews in cell biology, tissue culture, and embryology. With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

### Example 1

### Increased lysis of stem cells but not their differentiated cells by Natural Killer cells; De-differentiation or reprogramming activates NK cell cytotoxicity

The aim of this study is to demonstrate the increased lysis of stem cells but not their differentiated counterparts by the NK cells, and to determine whether disturbance in cell differentiation is a cause for increased sensitivity to NK cell mediated cytotoxicity. Increased cytotoxicity and augmented secretion of IFN-γ were both observed when PBMCs or NK cells were co-incubated with primary UCLA oral squamous carcinoma stem cells (UCLA-OSCSCs) when compared to differentiated UCLA oral squamous carcinoma cells (UCLA-OSCCs). In addition, human embryonic stem cells (hESCs) and human dental pulp stem cells (hDPSCs) were also lysed greatly by the NK cells. Moreover, NK cells were found to lyse human Mesenchymal Stem Cells (hMSCs), and human induced pluripotent stem cells (iPSCs) significantly more than their differentiated counterparts or parental lines from which they were derived. It was also found that inhibition of differentiation or reversion of cells to a less differentiated phenotype by blocking NFκB or targeted knock down of COX2 significantly augmented NK cell cytotoxicity and secretion of IFN-γ. Taken together these results demonstrate that stem cells are significant targets of the NK cell cytotoxicity, however, to support differentiation of stem cells, NK cells may be required to lyse a great number of stem cells and/or those which are either defective or incapable of full differentiation in order to lose their cytotoxic function and gain in cytokine secretion capacity (split anergy). Therefore, patients with cancer may benefit from repeated allogeneic NK cell transplantation for specific elimination of cancer stem cells.

Here we demonstrate that blocking NFκB in these cells increased the activation of NK cell cytotoxicity. We also used an immortalized but non tumorigenic oral keratinocytes HOK-16B since they were previously used as a model of dysplasia in a cancer progression model.

In this report we demonstrate that the stage of maturation and differentiation of the cells is predictive of their sensitivity to NK cell lysis. Thus, UCLA-OSCSCs, which are less differentiated oral tumors are significantly more susceptible to NK cell mediated cytotoxicity; however, their differentiated counterparts UCLA-OSCCs are significantly more resistant. In addition, both hESCs and iPSCs as well as a number of other stem cells such as hMSCs and hDPSCs were found to be significantly more susceptible to NK cell mediated cytotoxicity. Based on these results, it is found that NK cells can play a significant role in differentiation of the cells by providing critical cytokines. However, to drive differentiation, NK cells will have to first receive signals from undifferentiated stem cells or those which have disturbed or defective capabilities to differentiate in order to lose cytotoxicity and gain in cytokine producing phenotype. These alterations in NK cell effector function will ultimately aid in driving differentiation of a minor population of surviving healthy as well as transformed cells. In cancer patients since the majority of NK cells have lost cytotoxic activity, they cells may eventually contribute rather than halt the progression of cancer by not only driving the differentiation of tumor cells but more importantly, by allowing the growth and expansion of the pool of cancer stem cells.

### Materials and Methods

Cell Lines, Reagents, and Antibodies. RPMI 1640 supplemented with 10% FBS was used for the cultures of human and mouse NK cells and human PBMCs. UCLA-OSCCs and UCLA-OSCSCs were isolated from freshly resected tongue tumors, and were cultured in RPMI 1640 supplemented with 10% FCS. Recombinant IL-2 was obtained from NIH-BRB. The mouse and human NK and monocyte purification kits were obtained from Stem Cell Technologies (Vancouver, Canada). The anti- CD133 and CD44 were obtained from Miltenyi biotec (Auburn, Calif.). Antibody to CD90 was purchased from Pharmingen/BD (San Diego, Calif.). Antibodies for CD16 and B7H1 were purchased from ebiosciences (San Diego, Calif.). EGFR antibody (Erbitox) was purchased from UCLA pharmacy. The antibodies against p65 subunit of NFκB and pSTAT3 were purchased from Santa Cruz (Santa Cruz, Calif.). Blocking antibodies against CD126 were purchased from Biosource (Camarillo, Calif.).

Human Mesenchymal stem cells (hMSCs), human Embryonic Stem cells (hESCs), human Dental Pulp Stem cells (hDPSCs), human induced pluripotent stem cells (hiPSCs). hMSCs were obtained from Poietics, Cambrex Bio Science (Walkerville, Md.) and they were cultured in Mesenchymal Stem Cell Basal Medium (MSCBM) supplemented with Mesenchymal Cell Growth Supplement (MCGS) (Cambrex Bio Science Walkerville, Md.). The MSCs were differentiated into osteoblasts using Osteogenic differentiation media which comprises of Osteogenic Differentiation BulletKit® that contains Basal Medium and one Osteogenic SingleQuot Kit® also purchased from Cambrex Bio Science (Walkerville, Md.). Human Mesenchymal stem cells were cultured in Mesenchymal Stem Cell Basal Medium (MSCBM) with the growth supplements according to the manufacturer's recommendations. For the induction of osteogenesis, MSC were seeded at a density of (1×10⁴ cells/well) in Osteogenic media with the recommended supplements. Media was replaced every three days and the cells were used in the experiments when they were 80% confluent.

hDPSCs were isolated as described previously and they were cultured in complete DMEM supplemented with 10% FBS. DPSCs were differentiated using b-glycerophosphate, ascorbic acid and dexamethasone as reported previously. hESC line H9 and hiPSC line hiPSC18 were used in this study. H9 and hiPSC18 were used at passages 45-50. hESC and hiPSC were grown on irradiated mouse embryonic fibroblasts (MEFs) in DMEM/F12 supplemented with 20% Knockout serum replacement (Invitrogen), 1 mM glutamine, 1× nonessential amino acids (NEAA), and 4 ng/ml of bFGF as previously described. 2-mercaptoethanol (1 mM Sigma) and penicillin/streptomycin (Hyclone) were added to growing cultures. For coculture assays, cells were seeded at a density of 10⁵ cells/well on Matrigel (BD Sciences) in conditioned media. Neonatal human dermal fibroblasts (NHDF-iPSC parental fibroblast line from ATCC) were cultured in DMEM supplemented with 10% FBS, 1 mM glutamine, 1× NEAA and penicillin/streptomycin.

Purification of human and mouse NK cells and monocytes. PBMCs from healthy donors were isolated as described before. Briefly, peripheral blood lymphocytes were obtained after Ficoll-hypaque centrifugation and purified NK cells were negatively selected by using an NK cell isolation kit (Stem Cell Technologies, Vancouver, Canada). The purity of NK cell population was found to be greater than 90% based on flow cytometric analysis of anti-CD16 antibody stained cells. The levels of contaminating CD3+ T cells remained low, at 2.4% ±1%, similar to that obtained by the non-specific staining using isotype control antibody throughout the experimental procedures. The adherent subpopulation of PBMCs was detached from the tissue culture plates and monocytes were purified using isolation kit obtained from Stem Cell Technologies (Vancouver, Canada). Greater than 95% purity was achieved based on flow cytometric analysis of CD14 antibody stained monocytes. Written informed consents approved by UCLA Institutional Review Board (IRB) were obtained from the blood donors and all the procedures were approved by the UCLA-IRB.

Single cell preparations of mouse splenocytes were used to negatively select for mouse NK cells using mouse NK isolation kit purchased from Stem Cell Technologies (Vancouver, Canada). The purity of mouse NK cells were greater than 90% based on staining with NK1.1 and DX5 antibodies. Murine monocytes were purified from bone marrow using monocyte isolation kit obtained from Stem Cell Technologies (Vancouver, Canada). The purity of monocytes was greater than 90% based on staining with anti-CD14 antibody.

ELISA and Multiplex Cytokine Array kit: Single ELISAs were performed as described previously. Fluorokine MAP cytokine multiplex kits were purchased from R&D Systems (Minneapolis, Minn.) and the procedures were conducted as suggested by the manufacturer. To analyze and obtain the cytokine concentration, a standard curve was generated by either two or three fold dilution of recombinant cytokines provided by the manufacturer. Analysis was performed using the Star Station software.

Surface and DNA Staining and apoptosis assay: Staining was performed by labeling the cells with antibodies as described previously Jewett et al. (1997). J Immunol 159(10): 4815-22.

Western Blot. Treated and untreated cells were lysed in a lysis buffer containing 50mM Tris-HCL (pH 7.4), 150 mM NaCl, 1% Nonidet P-40 (v/v), 1 mM sodium orthovanadate, 0.5 mM EDTA, 10 mM NaF, 2 mM PMSF, 10 μg/mL leupeptin, and 2 U/mL aprotinin for 15 minutes on ice. The samples were then sonicated for 3 seconds. The cell lysates were centrifuged at 14;000 rpm for 10 minutes and the supernatants were removed and the levels of protein were quantified by the Bradford method. The cell lysates were denatured by boiling in 5× SDS sample buffer. Equal amounts of cell lysates were loaded onto 10% SDS-PAGE and transferred onto Immobilon-P membranes (Millipore, Billerica Mass.). The membranes were blocked with 5% non-fat milk in PBS plus 0.1% Tween-20 for 1 hour. Primary antibodies at the predetermined dilution were added for 1 hour at room temperature. Membranes were then incubated with 1:1000 dilution of horseradish peroxidase-conjugated secondary antibody. Blots were developed by enhanced chemiluminescence (ECL-purchased from Pierce Biotechnology, Rockford, Ill.).

⁵¹Cr release cytotoxicity assay. The ⁵¹Cr release assay was performed as described previously (Jewett, Wang et al. 2003). Briefly, different numbers of purified NK cells were incubated with ⁵¹Cr-labeled tumor target cells. After a 4 hour incubation period the supernatants were harvested from each sample and counted for released radioactivity using the gamma counter. The percentage specific cytotoxicity was calculated as follows;

$$\% Cytotoxicity = \frac{Experimental cpm - spontaneous cpm}{Total cpm - spontaneous cpm}$$

LU 30/10⁶ is calculated by using the inverse of the number of effector cells needed to lyse 30% of target cells ×100.

Retroviral and lentiviral transduction. UCLA-OSCCs were infected with culture supernatants of NIH 3T3 packaging cells transfected with either IκB_{(S32AS36A)}super-repressor or mutant IκBα (IκBαM) or their EGFP control vectors. The retroviral vectors were generated in Dr. Nicholas Cacalano's laboratory. Forty eight hours after infection the UCLA-OSCCs or HOK-16B cells were sorted for high expressing GFP cells and were grown and used in the experiments.

NFκB-Luciferae lentiviral reporter vector was produced by co-transfection of the packaging cell line 293T using Calcium Phosphate precipitation. UCLA-OSCCs and UCLA-OSCSCs were seeded at a density of 2×10⁵ cells per well in a 6-well culture plate 24 hrs before transduction. The following day, cells were transduced with the NFκB-Luciferase lentiviral reporter vector. To enhance transduction efficiency, the cationic polymer Polybrene was used at a final concentration of 8 μg/ml. After six hours of incubation, medium was re-freshed and transduced cells were incubated for an additional 42 hours. Cells were then harvested, lysed and luciferase activity was measured [RLU/s] using a luminometer. An internal lentiviral vector control constitutively expressing Luciferase was used to normalize values.

Luciferase reporter assay: Transfections were also performed using NFκB Luciferase reporter vector and Lipofectamine 2000 reagent (Invitrogen, Calif.) in Opti-MEM media (Invitrogen, Calif.) for 18 hours after which they were adhered to the plate overnight before different immune effectors at 1:1 Effector to target ratios were added. The cells were then lysed with lysis buffer and the relative Luciferase activity was measured using the Luciferase assay reagent kit obtained from Promega (Madison, Wis.).

Alkaline Phosphatase (ALP) staining. Human MSCs were co-cultured with and without untreated and IL-2 treated PBMCs as indicated in the result section. Cells were then washed twice with PBS and incubated with 120 mM of Tris buffer (pH=8.4) containing 0.9 mM Napthol AS-M Phosphate and 1.8 mM Fast Red TR (both purchased from Sigma, Mo.) for 30 minutes at 37° C. After 30 minute incubation, cells were washed three times with PBS and then fixed with 1 ml cold ethanol (100%) for 30 minutes. The stained cultures were scanned using an Epson scanner 1250.

Statistical analysis: An unpaired, two-tailed student t- test was performed for the statistical analysis. One way ANOVA with a Bonferroni post-test was used to compare the different groups.

Results

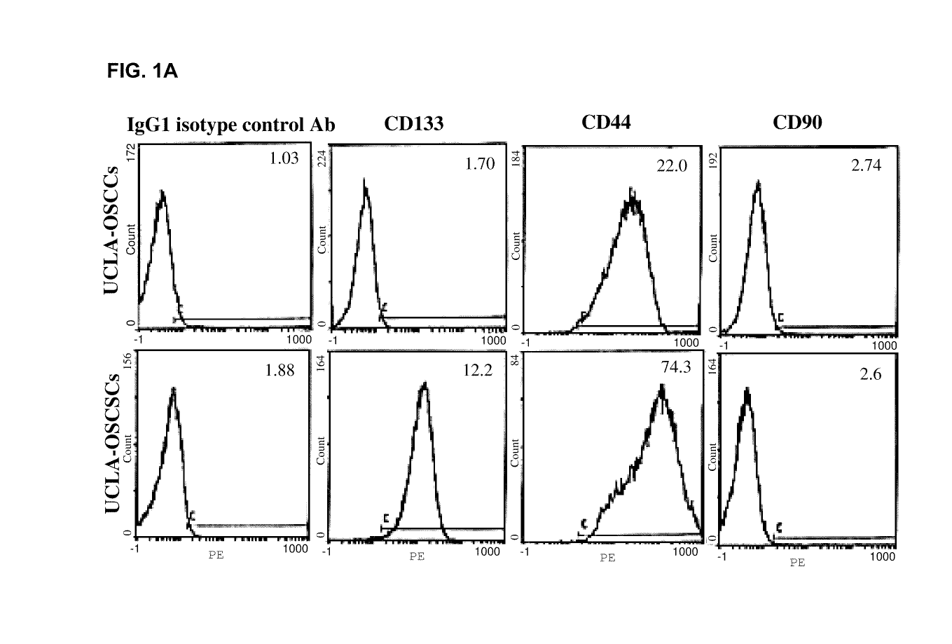

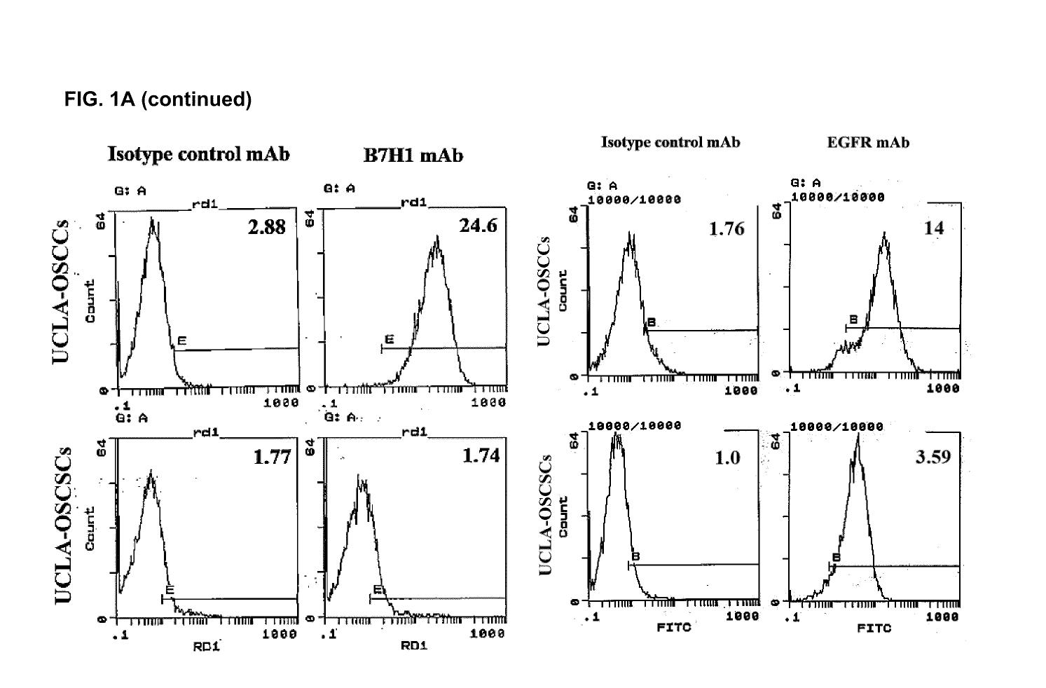

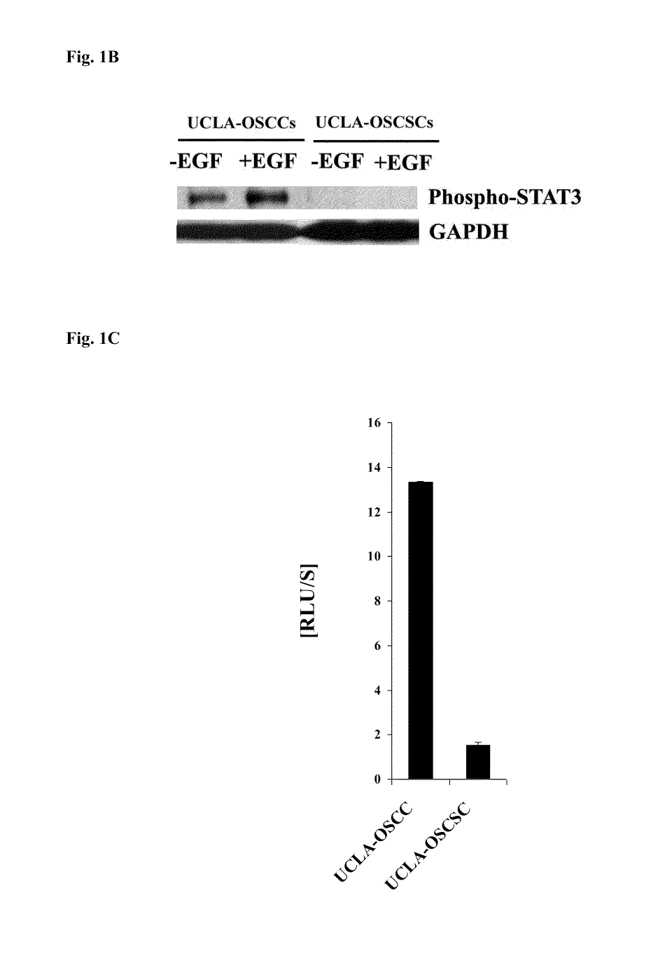

Identification and characterization of patient-derived primary oral squamous cancer stem cells (UCLA-OSCSCs). We screened a number of different primary oral squamous cell carcinomas (OSCC) derived from patients at UCLA, and selected to concentrate on two specific primary tumors based on their phenotypic characteristics and sensitivity to NK cell mediated cytotoxicity. UCLA-OSCCs were found to have higher surface expression of B7H1 and EGF-R and moderate expression of CD44 and no surface expression of CD133 whereas UCLA-OSCSCs expressed no or very low expression of B7H1, EGF-R and very high expression of CD133 and CD44^{bright}. No surface expression of MHC-Class II or CD90 could be seen on either tumor type (FIG. 1A). In addition; UCLA-OSCSCs secreted no or very low levels of IL-6, IL-8 and GM-CSF whereas they secreted higher levels of VEGF when compared to UCLA-OSCCs (Tables 1 and 2). Moreover, they did not express phospho-Stat3 when cultured in the presence and absence of EGF (FIG. 1B). More importantly, no or very low NFκB activity could be detected in UCLA-OSCSCs when compared to UCLA-OSCCs (FIG. 1C). Therefore, the profiles of cytokines secreted by UCLA-OSCCs and UCLA-OSCSCs resembled those of vector alone and IκB_{(S32AS36A)} super-repressor transfected HEp2 cells respectively (Table 2). Thus, UCLA-OSCSCs express phenotypic characteristics of oral cancer stem cells. Furthermore, they were smaller in size and proliferated at a much higher rate when compared to UCLA-OSCC cells. We used these two primary oral tumors to study NK cell activation.

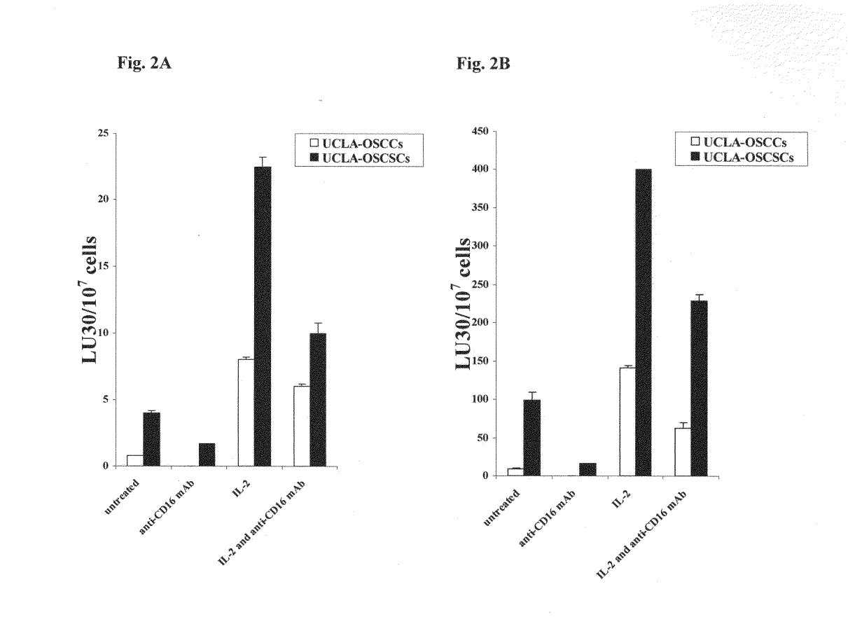

Increased NK cell cytotoxicity against UCLA-OSCSCs but not those of UCLA-OSCCs. We have previously shown that blocking NFκB in HEp2 tumor cells decreased IL-6 and IL-8 secretion substantially and resulted in an increased sensitivity of HEp2 tumor cells to NK cell mediated cytotoxicity. Therefore, using the levels of cytotoxicity, IFN-γ and IL-6 secretion, we could demonstrate a direct correlation between decreased IL-6 and increased IFN-γ secretion in the co-cultures of NK cells with NFκB knock down HEp2 cells and increased susceptibility to IL-2 activated NK cell killing. Induction of NK cell anergy by anti-CD16 antibody abrogated the ability of IL-2 treated NK cells to lyse HEp2 cells, even though the same treatment resulted in a significant induction of IFN-γ secretion in the co-cultures of NK cells with HEp2 cell transfectants. To extend our findings to patient derived oral tumors, UCLA-OSCC and UCLA-OSCSCs were tested for their sensitivity or resistance to NK cell mediated cytotoxicity. The cytotoxic activities of IL-2 treated PBMCs (FIG. 2A) and NK cells (FIG. 2B) were significantly higher against UCLA-OSCSCs cells when compared to UCLA-OSCCs. Untreated PBMCs or NK cells lysed UCLA-OSCSCs tumors significantly more than UCLA-OSCCs (FIG. 2B). However, the levels of lysis by untreated NK cells were considerably lower than that obtained by IL-2 treated PBMCs or NK cells (FIG. 2A and 2B). Treatment of PBMCs or NK cells with anti-CD16 mAb decreased cytotoxicity significantly against both tumor types, however, the levels of lysis by the NK cells remained higher against UCLA-OSCSCs in all the NK samples tested (FIG. 2). IL-2 treated NK cells co-cultured with UCLA-OSCSCs oral tumor cells exhibited higher expression of CD69 activation antigen when compared to those co-cultured with UCLA-OSCC oral tumors.

Increased induction of IFN-γ was paralleled with a decreased secretion of IL-6 in co-cultures of NK cells with UCLA-OSCSCs oral tumors. Untreated and IL-2 treated NK cells were co-cultured with UCLA-OSCC and UCLA-OSCSCs and the induction of a number of key cytokines, including those which were correlated with NK resistant tumor phenotype, were determined in the supernatants recovered from the co-cultures of the immune effectors with oral tumors after an overnight incubation. In the presence of untreated NK cells co-cultured with UCLA-OSCC, synergistic induction of GM-CSF, IL-6 and IL-8 could be observed since much lower levels of these cytokines were induced either in the presence of immune effectors alone or tumor cells alone (Table 2). The levels of above-mentioned cytokines were considerably lower in the co-cultures of untreated NK cells with UCLA-OSCSCs (Table 2). VEGF secretion was significantly higher in UCLA-OSCSCs, the levels exceeded that of the baseline levels produced by the tumor cells alone when untreated NK cells were co-cultured with UCLA-OSCC cells and not that of UCLA-OSCSCs (Table 2). Increased GM-CSF secretion in the presence of UCLA-OSCCs as compared to UCLA-OSCSCs was more evident in untreated NK cells (Table 2).

NK cell sensitivity of tumors correlated with an increased IFN-γ secretion in the presence of lower IL-6 and IL-8 secretion in IL-2 activated NK cells co-cultured with UCLA-OSCSCs (Table 2). Indeed, when ratios of IL-6 to IFN-γ were considered a direct correlation between sensitivity to NK cell mediated killing and decreased ratios of IL-6 to IFN-γ could be seen (Table 2). Finally, both cell lines exhibited lower amounts of VEGF secretion in the presence of IL-2 treated NK cells, indicating the ability of IL-2 treated NK cells to exert significant inhibitory effect on VEGF secretion. However, the residual levels remained higher in the co-cultures of IL-2 treated NK cells with UCLA-OSCC than UCLA-OSCSCs when compared to the baseline secretion by the tumors alone (Table 2). Thus, several important cytokine profiles were identified for NK sensitive and resistant oral tumors after their co-culture with NK cells.

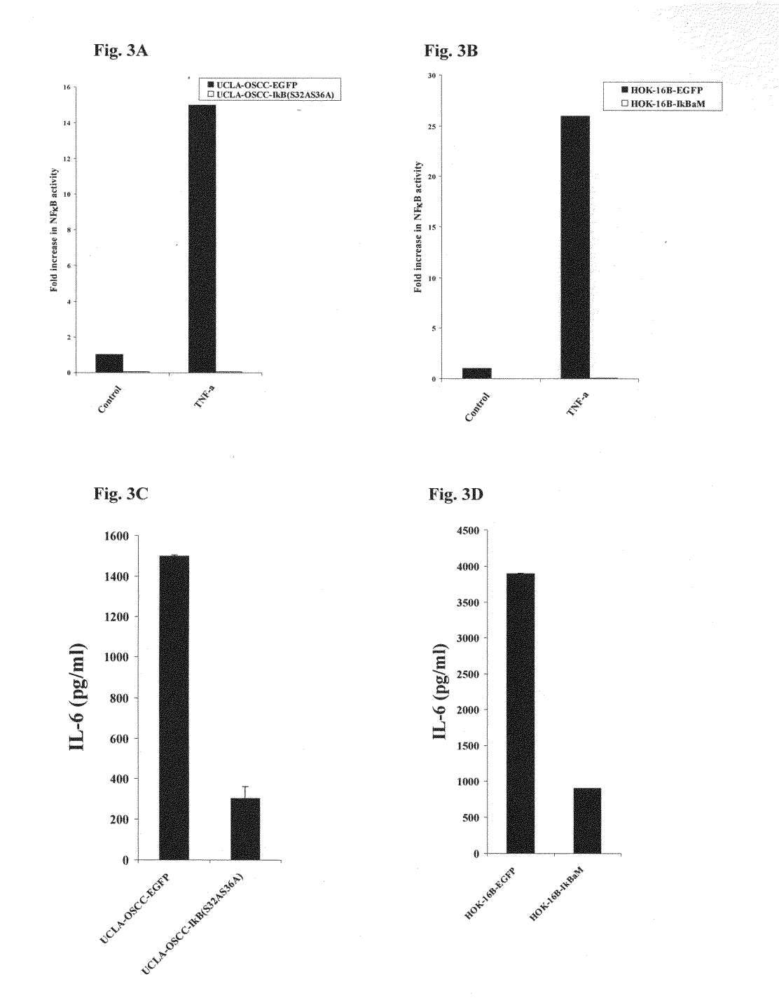

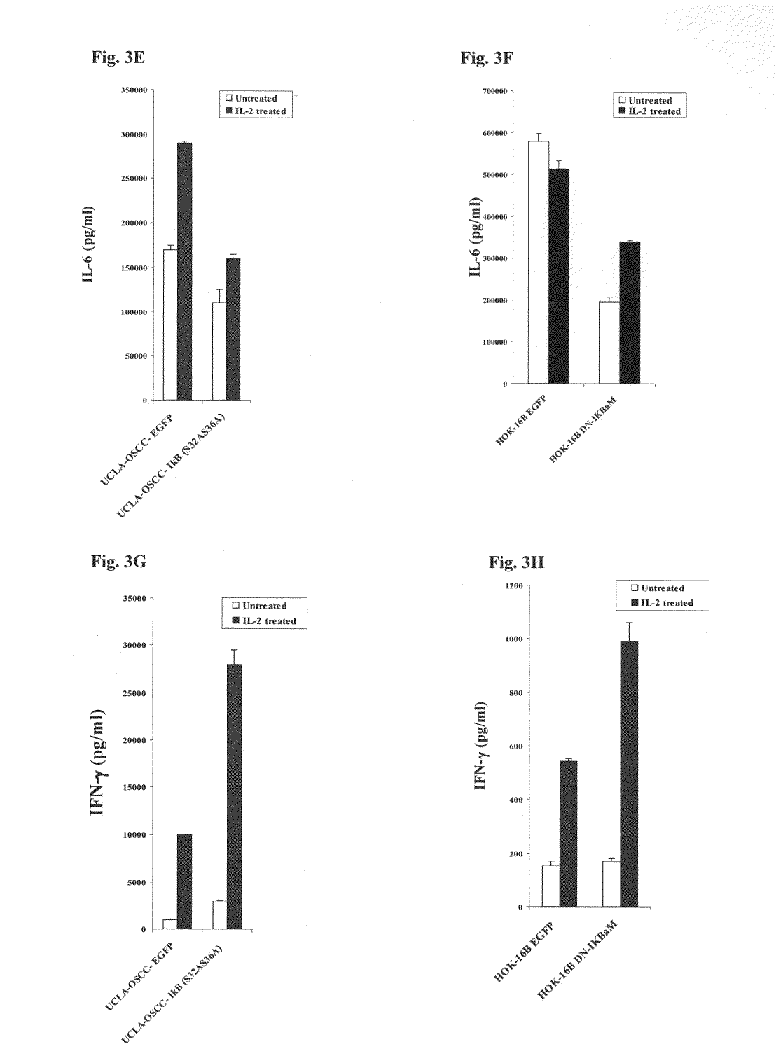

Blocking NFκB in UCLA-OSCCs and HOK-16B oral epithelial cells lowered IL-6 to IFN-γ ratios and increased their sensitivity to NK cell mediated cytotoxicity. As indicated previously UCLA-OSCCs and HOK-16B oral keratinocytes represent an oral cancer progression model since HOK-16B are immortalized but non-tumorigenic, and thus could represent a model of dysplastic keratinocytes. HOK-16B and UCLA-OSCCs were transduced with EGFP alone or IκBαM or IκB_{(S32AS36A)}super-repressor retroviral constructs and sorted for high GFP expressing cells using flow cytometry. The inhibition of NFκB by the IκBαM or IκB_{(S32AS36A)}super-repressor retroviral vector in UCLA-OSCC and HOK-16B was confirmed by measuring NFκB activity using luciferase reporter assay (FIGS. 3A and 3B). IκBαM or IκB_{(S32AS36A)}super-repressor transduced UCLA-OSCC (FIG. 3C) and HOK-16B (FIG. 3D) tumor cells secreted substantially lower levels of IL-6 when compared to EGFP transduced UCLA-OSCCs and HOK-16B cells. Thus, transduction of UCLA-OSCCs and HOK-16B with IκBαM or IκB_{(S32AS36A)}super-repressor constructs exhibited the same functional profiles as those observed in transfected HEp2 oral tumor cells with IκB_{(S32AS36A)} construct. Similar to HEp 2-cell transfectants, UCLA-OSCCs and HOK-16B cells transduced with IκBαM or IκB_{(S32AS36A)}super-repressor constructs did not exhibit elevated levels of cell death when assessed by flow cytometric analysis of Annexin V and PI stained cells. In addition, there was a significant decrease in the surface expression of ICAM-1 in IFN-γ treated IκB_{(S32AS36A)} transduced UCLA-OSCCs (83% decrease) and HOK-16B cells (78% decrease) when compared to EGFP alone transduced cells. These results also indicated that IL-6 secretion in oral tumor cells is regulated by the function of NFκB.

Untreated or IL-2 treated NK cells were added to EGFP or IκB_{(S32AS36A)} transduced UCLA-OSCCs and IκBαM transduced HOK-16B oral keratinocytes and the levels of IL-6 and IFN-γ secretion were determined in the co-cultures with the NK cells after an overnight incubation. IL-2 activated NK cells secreted lower levels of IL-6 when co-cultured with IκB_{(S32AS36A)} transduced UCLA-OSCCs (FIG. 3E) and IκBαM HOK-16B (FIG. 3F) cells as compared. to EGFP transduced oral keratinocytes. In contrast, higher induction of IFN-γ secretion could be observed in supernatants recovered from the co-cultures of NK cells with IκB_{(S32AS36A)} transduced UCLA-OSCCs (FIG. 3G) and IκBαM transduced HOK-16B (FIG. 3H) oral keratinocytes as compared to EGFP transduced oral tumors. Similar NK cell response patterns were obtained when NFκB was inhibited in HEp2 cells. Finally, IL-2 treated NK cells lysed NFκB knock down OSCCs (FIG. 31) and HOK-16B (FIG. 3J) cells significantly more than EGFP transfected cells.

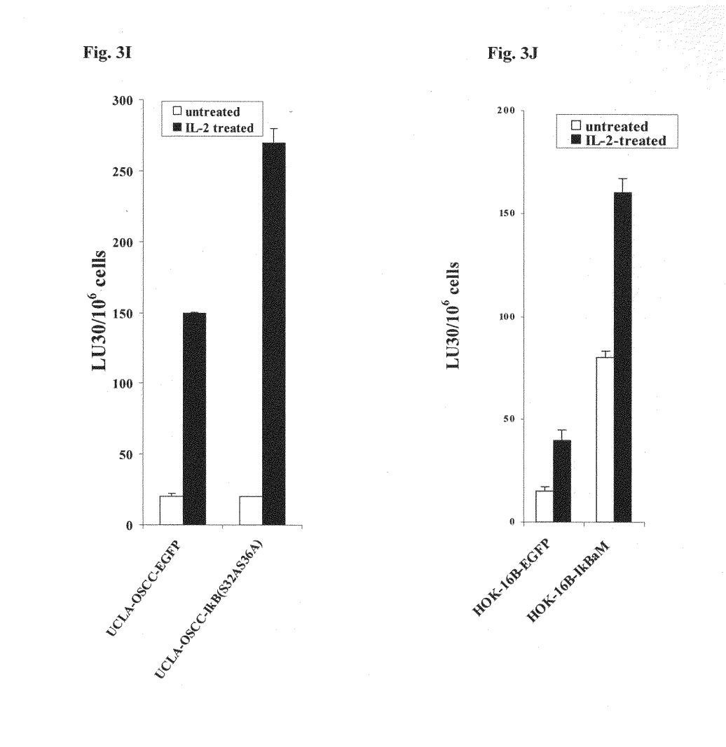

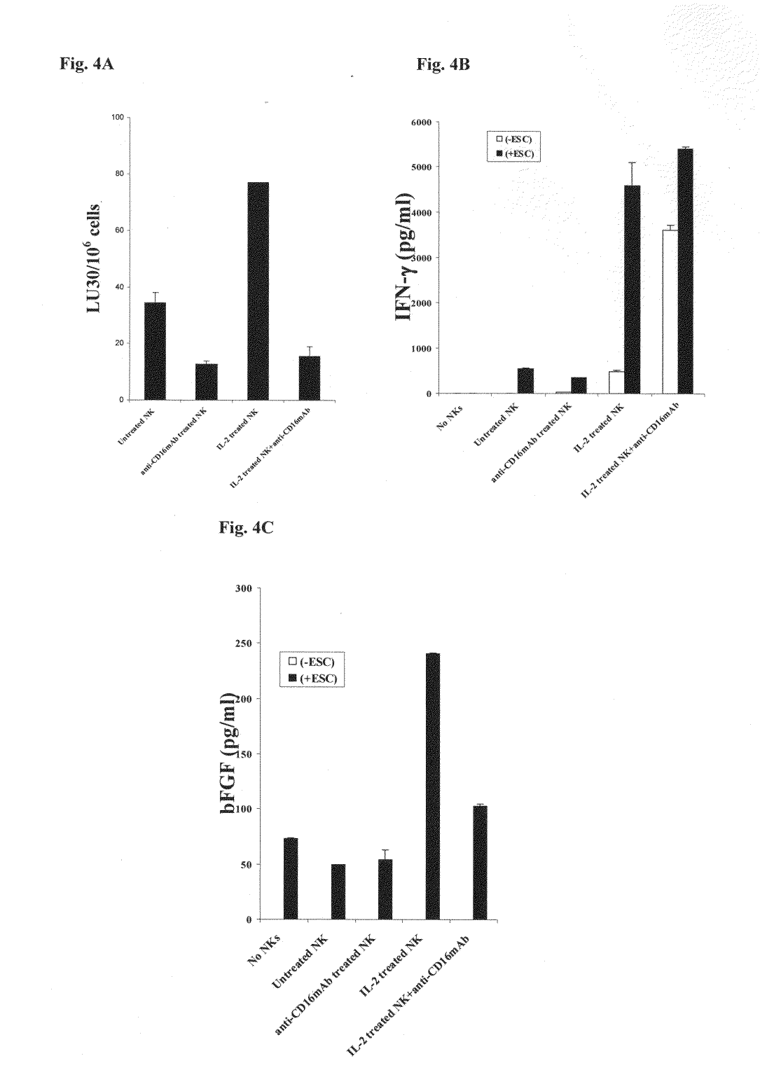

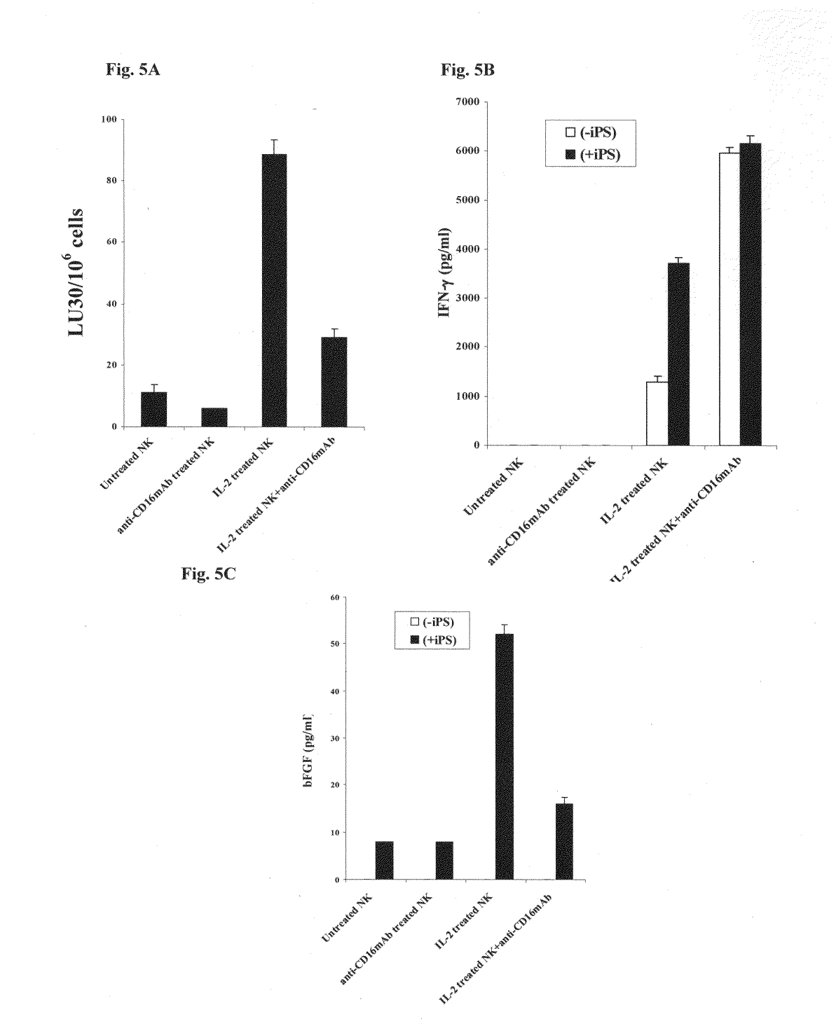

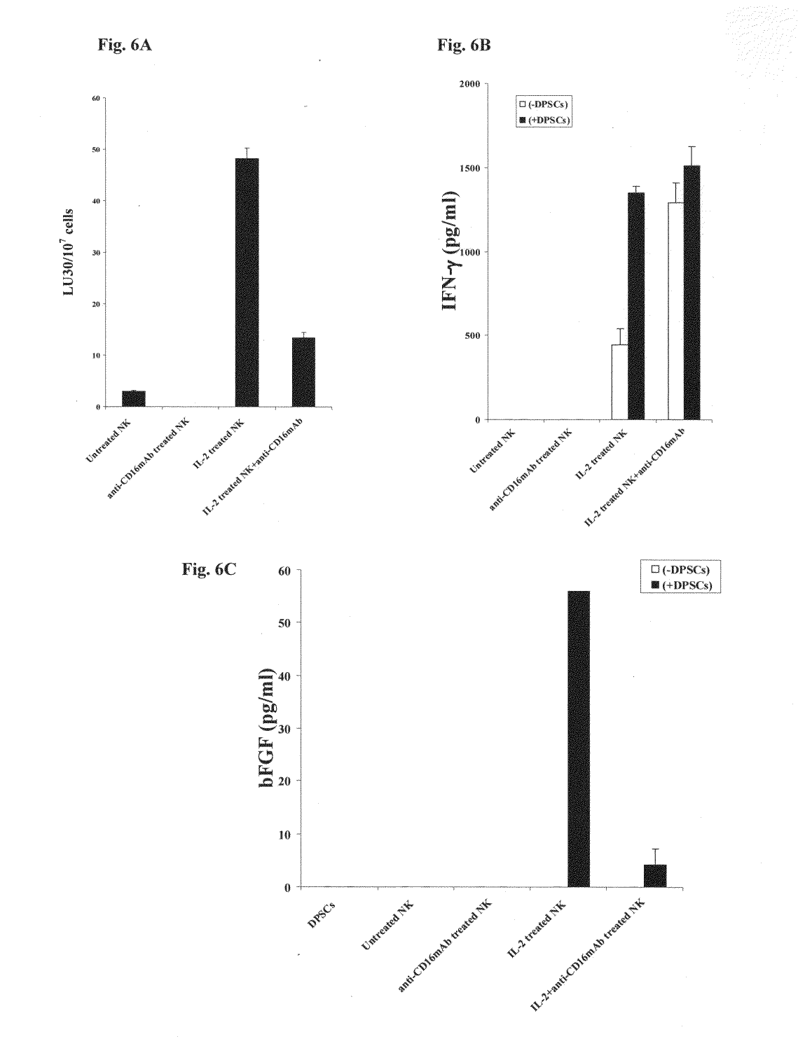

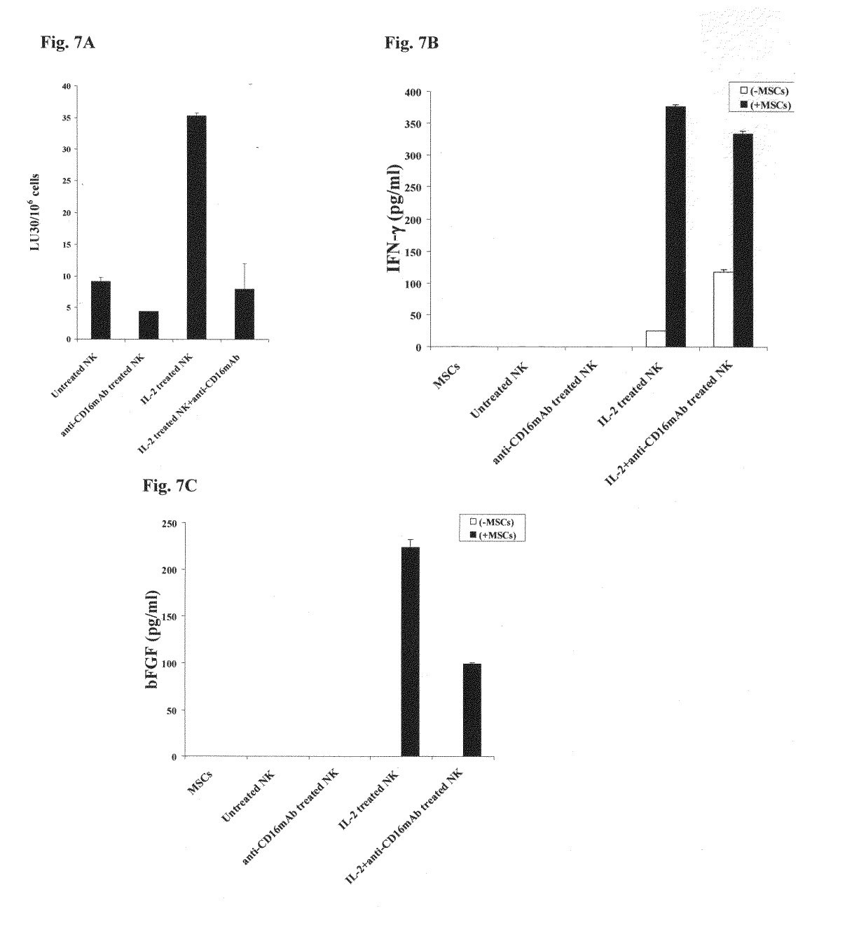

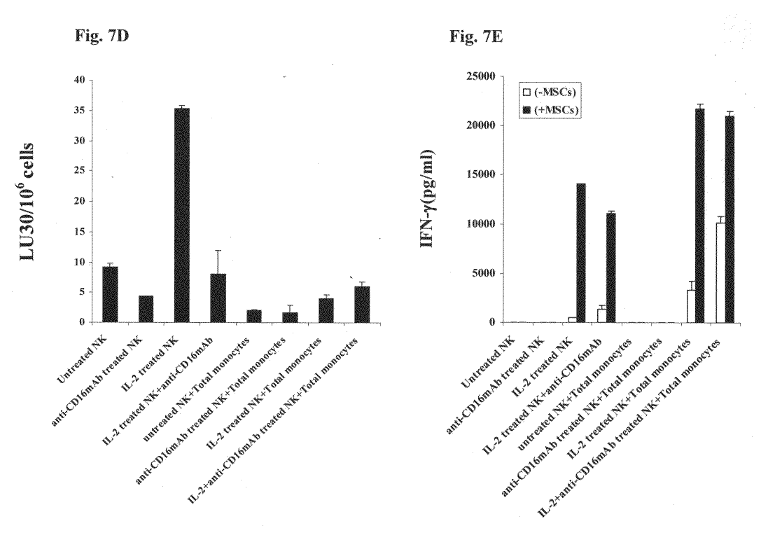

Significant lysis of Embryonic Stem Cells (hESCs), Induced Pluripotent Stem Cells (iPS), Dental Pulp Stem Cells (DPSCs), and Mesenchymal Stem Cells (MSCs) by untreated or IL-2 treated NK cells. Highly purified human NK cells were cultured with-and without IL-2 for 12-24 hours before they were added to ⁵¹Cr labeled hESCs (FIG. 4A), iPSCs (FIG. 5A), DPSCs (FIG. 6A) and MSCs (FIG. 7A). Addition of untreated NK cells had lower cytotoxicity against different populations of stem cells whereas activation with IL-2 increased cytotoxicity against all stem cell populations significantly (p<0.05) (FIGS. 4A-7A). Therefore, human stem cells are greatly lysed by the NK cells.

Lysis of hESCs, iPSCs, DPSCs, and MSCs by untreated and IL-2 treated NK cells is inhibited by anti-CD16 antibody treatment, however, the same treatment induced significant secretion of IFN-γ by the NK cells in the presence and absence of stem cells. As shown in a number of previous studies anti-CD16 mAb treatment induced anergy in a great majority of the NK cells as well as it induced death in a subset of NK cells, thereby inhibiting NK cell cytotoxicity against different populations of stem cells (p<0.05) (FIGS. 4A-7A). Addition of the combination of IL-2 and anti-CD16 treatment also induced anergy and NK cell death and inhibited NK cell cytotoxicity against stem cells when compared to IL-2 activated NK cells (p<0.05) (FIG. 4A-7A). Untreated or anti-CD16 mAb treated NK cells did not secrete IFN-γ when co-cultured with any of the stem cell populations, however, both IL-2 treated and IL-2 in combination with anti-CD16 mAb treated NK cells in the presence and absence of stem cells secreted significant levels of IFN-γ (p<0.05) (FIG. 4B-7B). Indeed, stem cells triggered significant secretion of IFN-γ from IL-2 treated NK cells when compared to IL-2 treated NK cells in the absence of stem cells. In addition, there was a synergistic induction of IFN-γ secretion in IL-2 and anti-CD16 mAb treated NK cells in the absence of stem cells, and the levels either remained the same or exceeded those in the absence of stem cells when IL-2 and anti-CD16 mAb treated NK cells were cultured with stem cells (FIG. 4B-7B). There was a direct correlation between secretion of bFGF by stem cells and cytotoxicity by IL-2 and IL-2+ anti-CD16 treated NK cells (FIG. 4C-7C).

Lysis of MSCs by untreated and IL-2 treated NK cells is inhibited by monocytes, however, the addition of monocytes induced significant secretion of IFN-γ by the NK cells in the presence and absence of stem cells. Monocytes were purified from PBMCs and irradiated as indicated in the Material and Methods section. MSCs were co-cultured with irradiated monocytes for 24-48 hours before they were labeled with ⁵¹Cr and used in the cytotoxicity assays against NK cells. NK cells were left untreated or pre-treated with anti-CD16 antibody and/or IL-2 for 24-48 hours before they were used in the cytotoxicity assays against MSCs. The addition of monocytes to MSCs significantly protected the MSCs (FIG. 7D) from NK cell mediated cytotoxicity (p<0.05). Significant inhibition of NK cell cytotoxicity by monocytes could be observed against untreated and IL-2 treated NK samples (p<0.05) (FIG. 7D). Monocytes also increased the levels of alkaline phosphatase staining in MSCs and prevented decrease in alkaline phosphatase expression induced by IL-2 activated NK cells. Untreated or anti-CD16 antibody treated irradiated monocytes did not mediate cytotoxicity against MSCs. Overall, these experiments indicated that monocytes protect MSCs against NK cell mediated lysis.

As expected IL-2 treated NK cells secreted moderate amounts of IFN-γ which were synergistically increased when co-cultured in the presence of MSCs (p<0.05) (FIG. 7E). The addition of anti-CD16 mAb in combination with IL-2 to NK cells in the absence of MSCs increased secretion of IFN-γ when compared to IL-2 alone treated NK cells in the absence of MSCs. IFN-γ secreted levels remained similar between IL-2 alone and IL-2 and anti-CD16 mAb treated NK cells cultured with MSCs (FIG. 7E). Monocytes added to IL-2 or IL-2 and anti-CD16 antibody treated NK cells in the absence of MSCs or those in the presence of MSCs, synergistically increased the levels of secreted IFN-γ (p<0.05) (FIG. 7E). However, the highest increase in IFN-γ release was seen when monocytes were added to IL-2 or IL-2 and anti-CD16 mAb treated NK cells with MSCs (FIG. 7E). These results indicated that monocytes increased IFN-γ in co-cultures with MSCs, and further synergized with IL-2 or IL-2 and anti-CD16 mAb treated NK samples to increase the release of IFN-γ in the co-cultures of NKs and MSCs. Similar results were obtained when NK cells were co-cultured with monocytes and DPSCs.

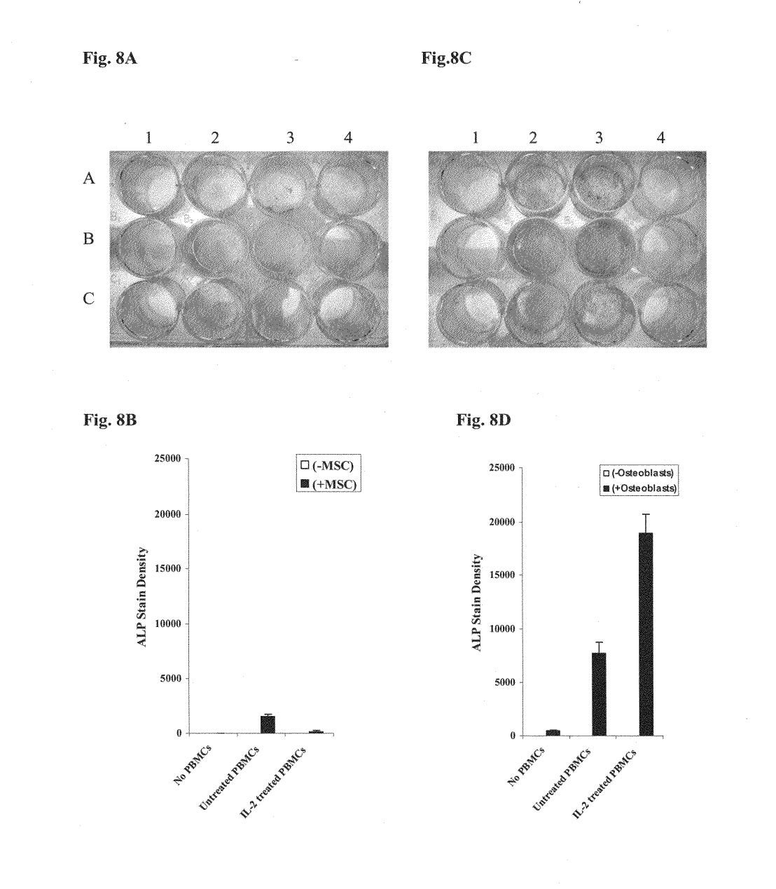

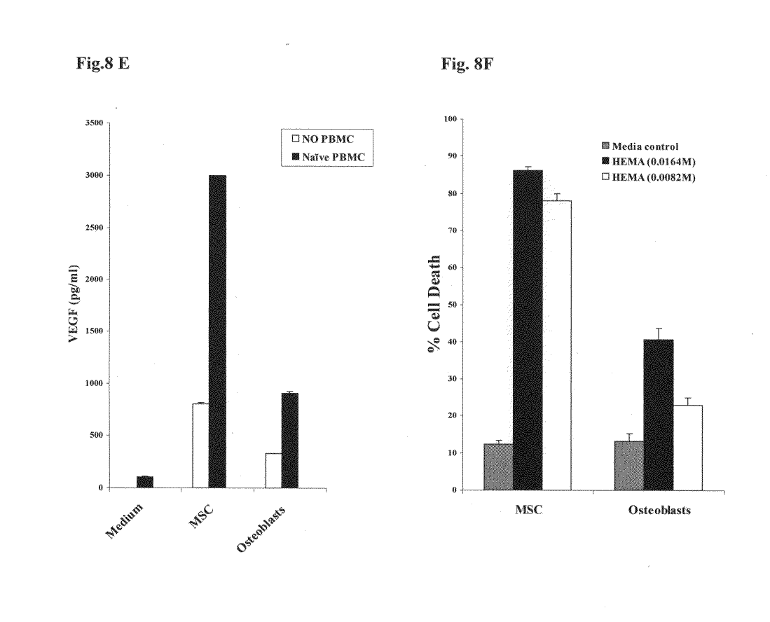

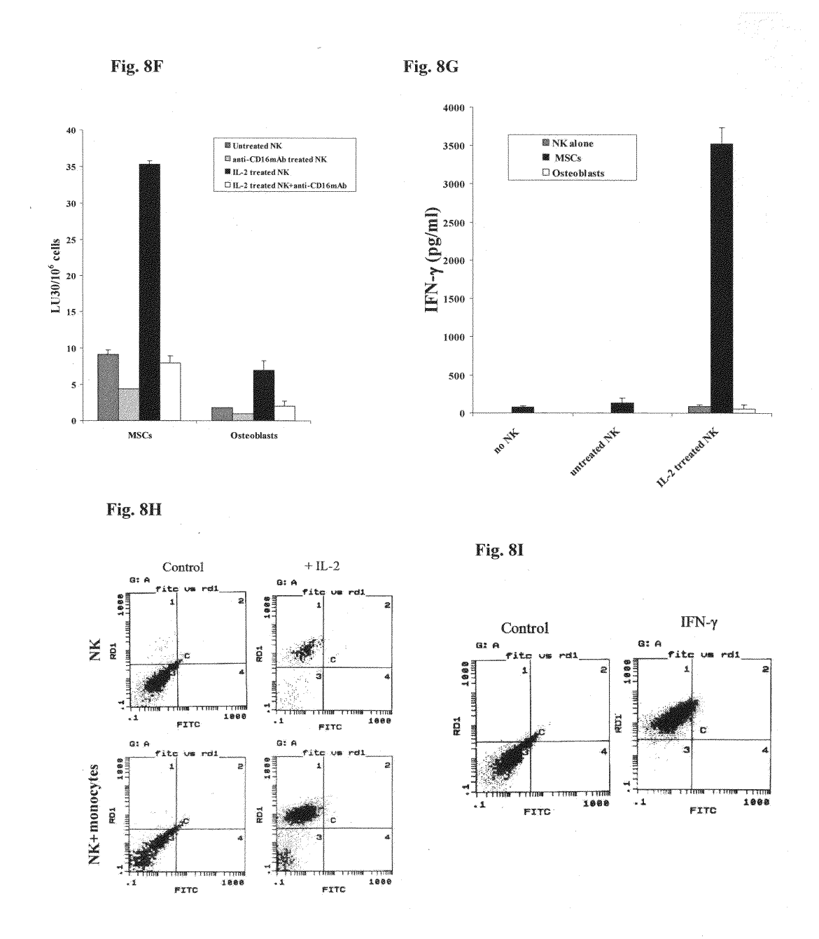

MSCs are significantly more sensitive to lysis by IL-2 treated NK cells than their differentiated counterparts and they trigger significant release of IFN-γ by IL-2 activated NKs. To determine whether differentiation decreases sensitivity of stem cells to NK cell mediated cytotoxicity we chose to concentrate on MSCs. To assess whether differentiation of MSCs similar to oral tumors decreases sensitivity of these cells to NK cell mediated cytotoxicity we determined NK cell cytotoxicity against MSCs and their differentiated osteoblasts using unfractionated PBMCs as well as NK cells. MSCs were cultured in the absence and presence of untreated and IL-2 treated PBMCs at 10:1 PBMC to MSC ratio and the levels of ALP staining were determined after 2 days of incubation. The addition of untreated PBMCs to MSCs triggered some differentiation of MSCs as assessed by Alkaline Phosphatase (ALP) staining (FIGS. 8A and 8B). No significant staining with ALP can be seen by either the PBMCs or MSCs alone (FIGS. 8A and 8B). Treatment of PBMCs with IL-2 and their subsequent co-culture with MSCs lysed the cells and prevented induction of ALP, therefore, no or very low detection of ALP could be observed (FIGS. 8A and 8B). The co-culture of differentiated osteoblasts with PBMCs was performed as described above with MSCs. As shown in FIGS. 8C and 8D both the untreated and IL-2 treated PBMCs triggered significant increase in ALP staining in osteoblasts. IL-2 treated PBMCs triggered much higher levels of ALP staining when compared to untreated PBMCs (FIGS. 8C and 8D). The levels of ALP staining in osteoblasts were substantially lower in the absence of PBMCs and no significant ALP staining can be seen in untreated or IL-2 treated PBMCs in the absence of osteoblasts (FIGS. 8C and 8D). These results indicated that stem cells were sensitive to lysis by IL-2 treated PBMCs whereas their differentiated counterparts were more resistant and unlike stem cells they were able to resist death and further upregulate ALP when cultured with IL-2 treated PBMCs. In addition, when the levels of VEGF secretion were determined higher induction of VEGF secretion by MSCs could be observed when compared to osteoblasts (FIG. 8E).

To further demonstrate the resistance of osteoblasts to cell death, MSCs and those differentiated to osteoblasts were also cultured in the absence and presence of different concentrations of HEMA and the levels of cell death were determined after an overnight incubation. Shown in FIG. 8F, HEMA induced significant cell death in undifferentiated MSCs when compared to differentiated osteoblasts. In addition, undifferentiated MSCs were significantly more sensitive to lysis by IL-2 treated NK cells when compared to their differentiated counterparts (FIG. 8G), and triggered significant secretion of IFN-γ in co-cultures with IL-2 treated NK cells (FIG. 8H). Moreover, when MSCs were cultured with NK cells alone or NK cells with monocytes significant induction of B7H1 surface expression could be observed in surviving MSCs (FIG. 8I). We have recently reported that monocytes protect stem cells from NK cell mediated lysis. Accordingly, significantly more surviving MSCs was observed in co-cultures with NK cells and monocytes than with NK cells alone. Monocytes alone were not able to elevate B7H1 expression on the surface of MSCs. The intensity of NK cell induced B7H1 expression on MSCs were similar to that induced by treatment of MSCs with IFN-γ (FIG. 8J). Thus, these results suggested that sensitivity of MSCs to cell death and to NK cell mediated cytotoxicity correlated with the degree of differentiation of these cells. Moreover, NK cells may contribute to differentiation and resistance of MSCs by increased induction of key resistance factors such as B7H1.

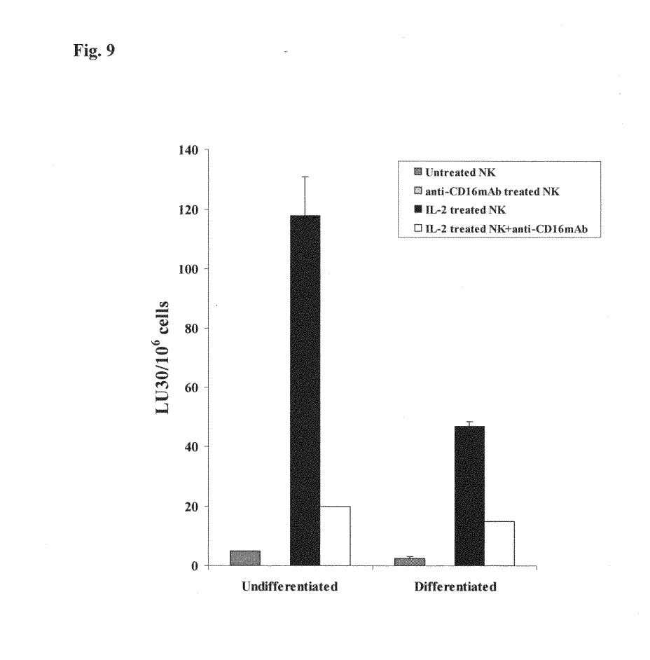

Differentiated DPSCs are more resistant to NK cell mediated cytotoxicity. DPSCs were differentiated by the addition of β-glycerophosphate, ascorbic acid and dexamethasone as reported previously, and NK cell cytotoxicity were determined against both the differentiated and undifferentiated DPSCs. As shown in FIG. 9 significantly less NK cell cytotoxicity could be obtained against differentiated DPSCs by untreated, IL-2 treated and IL-2 plus anti-CD16 mAb treated NK cells when compared to undifferentiated DPSCs. In addition, significantly less NK cell cytotoxicity could be seen against passage 8 when compared to passage 3 undifferentiated DPSCs. Therefore, a stepwise decrease in NK cell cytotoxicity could be observed depending on the stage of the differentiation of DPSCs.

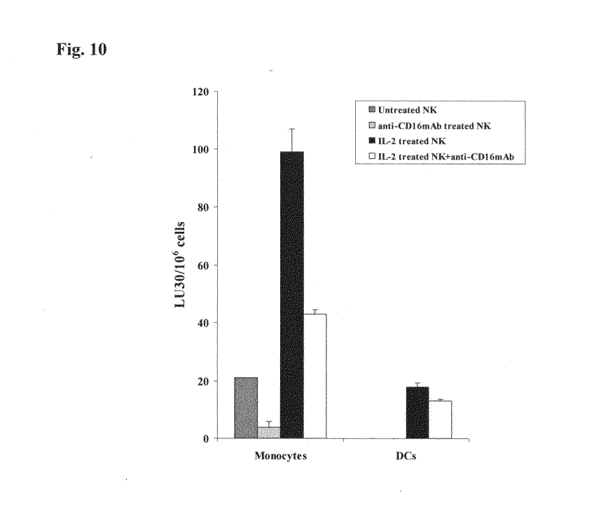

Decreased sensitivity of dendritic cells to NK cell mediated lysis. To demonstrate that resistance of NK cell mediated cytotoxicity by increased differentiation of stem cells is not restricted to only certain types of cells, we used monocytes and their differentiated counterpart dendritic cells to determine sensitivity to NK cell mediated lysis. We have recently shown that monocytes have exquisite sensitivity to NK cell mediated lysis. As shown in FIG. 10 monocytes were significantly more sensitive to NK cell mediated cytotoxicity than DCs, their differentiated counterparts.

IPS cells are more susceptible to NK cell mediated cytotoxicity than their parental line. Since more differentiated cells were less sensitive to NK cell mediated lysis, we aimed at characterizing the sensitivity of iPS cells as well as the parental line from which they were derived to NK cell mediated lysis. As shown in FIG. 11 untreated or IL-2 treated NK cells lysed iPS cells significantly more than the parental line. Treatment of NK cells with anti-CD16 mAb or a combination of IL-2 and anti-CD16 mAb decreased cytotoxicity mediated by the NK cells (FIG. 11). Therefore taken together the results shown thus far suggest that any attempt in reprogramming or de-differentiating the cells may result in increased sensitivity of the cells to NK cell mediated lysis. We, therefore, performed additional experiments using mice which had targeted knock down of COX2 gene in myeloid subsets to determine whether blocking COX2 which is shown to be elevated in many tumors and is important in differentiation of the cells can elevate sensitivity to NK cell mediated lysis.

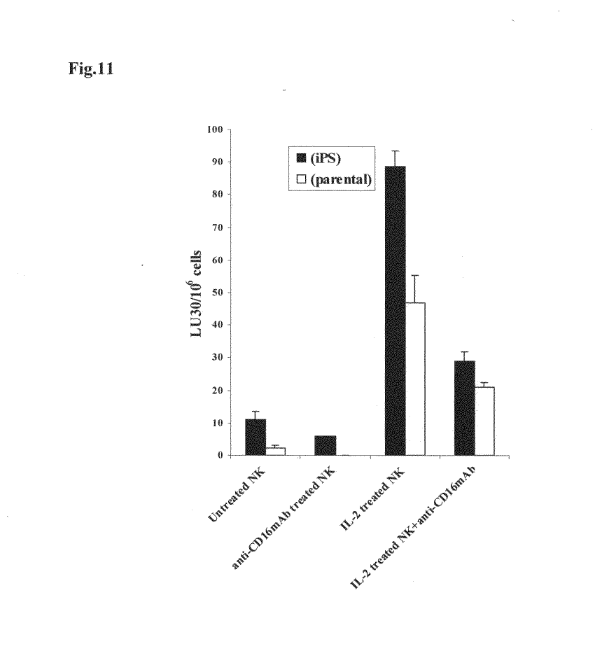

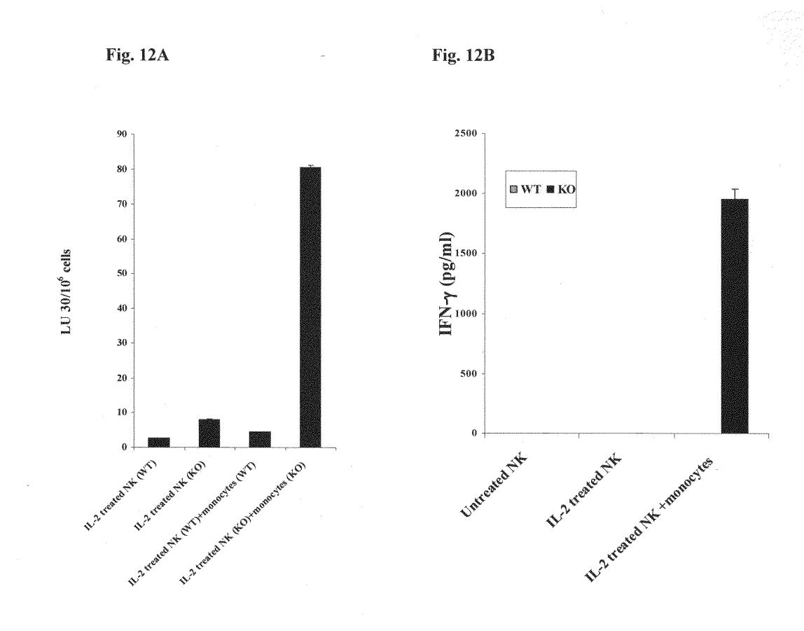

Targeted inhibition of COX2 in bone marrow derived monocytes from LysMCre+/− mice increased cytotoxicity and secretion of IFN-γ by IL-2 treated NK cells. Purified NK cells obtained from spleens of control mice and those with targeted knock down of COX2 gene in myeloid cells were cultured with and without bone marrow derived purified monocytes for 6 days before they were added to ⁵¹Cr YAC cells and cytotoxicity were determined in 4 hours ⁵¹Cr release assay. As shown in FIG. 12A NK cells purified from Cox-2flox/flox LysMCre/+ mice and cultured with autologous COX2−/− monocytes lysed YAC cells significantly more, whereas NK cells from control mice (Cox-2flox/flox LysM+/+) cultured with autologous COX2+/+ monocytes had very little cytotoxicity. Similarly, NK cells purified from Cox-2flox/flox LysMCre/+ mice and cultured with autologous COX2−/− monocytes secreted higher levels of IFN-γ when compared to NK cells from control mice (Cox-2flox/flox LysM+/+) cultured with autologous COX2+/+ monocytes (FIG. 12B).

We have characterized the interaction of a number of oral tumors and a transformed but non-tumorigenic oral keratinocyte line with NK cells and identified several important profiles that could distinguish between differentiated NK resistant oral tumors from undifferentiated NK sensitive tumor stem cells. The results also indicated that the level of NK cell cytotoxicity may vary depending on the expression and function of NFκB in tumors. Thus, increased NFκB appears to be an important factor of differentiation, survival and function of primary oral tumors during their interaction with NK cells.

Increased NK cell cytotoxicity and augmented secretion of IFN-γ were observed when NK cells were co-incubated with UCLA-OSCSCs which released significantly lower levels of GM-CSF, IL-6 and IL-8 and demonstrated decreased expression of phospho-Stat3, B7H1 and EGFR, and much lower constitutive NFκB activity when compared to differentiated UCLA-OSCCs. More importantly, UCLA-OSCSCs expressed oral stem cell marker CD133 and CD44^{bright}. Addition of untreated fresh NK cells to UCLA-OSCCs, which were unable to lyse the tumor cells, synergistically contributed to the elevation of the above mentioned cytokines in the co-cultures of NK cells with UCLA-OSCCs. In contrast, untreated NK cells, which lysed UCLA-OSCSCs, were either unable to increase or moderately increased the secretion of resistant factors in the co-cultures of NK cells with UCLA-OSCSCs. Untreated NK cells increased the secretion of VEGF in NK-UCLA-OSCC co-cultures whereas a decrease in VEGF secretion was observed in NK− UCLA-OSCSCs co-cultures when compared to those secreted by the tumors. alone. Although the majority of secreted cytokines tested were elevated in UCLA-OSCCs when compared to UCLA-OSCSCs, the levels of VEGF secretion were higher in UCLA-OSCSCs when compared to UCLA-OSCCs. This observation is in agreement with the previously published results where decreased expression of VEGF was seen during the progression of head and neck tumors.

Increase in IFN-γ secretion correlated with a decrease in secretion of IL-6 in the co-cultures of NK cells with UCLA-OSCSCs when compared to UCLA-OSCCs. Furthermore, the potent function of IL-2 activated NK cells could also be seen in regards to suppression of VEGF secretion in tumor cells. Therefore, from these results a specific profile for NK resistant oral tumors emerged which demonstrated increased GM-CSF, IL-6 and IL-8 secretion in the context of decreased IFN-γ secretion during their interaction with the NK cells. In contrast, co-cultures of cancer stem cells with NK cells demonstrated increased IFN-γ in the context of lower GM-CSF, IL-6 and IL-8 secretion.

Many aggressive and metastatic tumor cells exhibit constitutively elevated NFκB activity. Similar to HEp2 cells blocking NFκB in UCLA-OSCCs and HOK-16B cells increased IFN-γ secretion and augmented the cytotoxic function of IL-2 activated NK cells against these cells (FIG. 6). Inhibition of NFκB in UCLA-OSCCs and HOK-16B was confirmed by several observations. First, the synergistic induction of ICAM-1 by TNF-α and IFN-γ treatment, which was previously shown to be due to increased function of NFκB, was greatly abrogated when UCLA-OSCCs and HOK-16B cells were transduced with IκB super-repressor. Second, significant decrease in IL-6 secretion could be observed in both cells and in the co-cultures of immune effectors with UCLA-OSCCs and HOK-16B cells transduced with IκB super-repressor. Lastly, decreased binding of NFκB was observed using luciferase reporter assay in NFκB knock down cells. Therefore, the profiles of NFκB knock down cells resembled those of undifferentiated UCLA-OSCSCs cells based on the parameters tested.

It appears that NFκB in primary oral keratinocytes may serve as the master molecular switch between IL-6 and IFN-γ secretion in the co-cultures of NK cells with tumors. IL-6 is secreted constitutively by oral squamous cell carcinomas and it is found to be elevated in oral cancer patients. IL-6 is known to interfere with IFN-γ signaling by the induction of Th2 differentiation via activation of NFAT which subsequently inhibits Th1 polarization. IL-6 is also known to induce Stat3 activation. Since blocking Stat3 function in tumor cells is also known to activate adaptive immunity (Morrison, Park et al. 2003; Wang, Niu et al. 2004) it may be that IL-6 induced Stat3 is in part responsible for the induction of NK cell inactivation and cell death in the co-cultures of NK cells and either HEp2 cells or UCLA-1 or HOK-16B tumors.

Since UCLA-OSCSCs were significantly more susceptible to NK cell mediated cytotoxicity we hypothesized that stem cells in general may also be more susceptible to NK cell mediated cytotoxicity. Indeed, we have recently demonstrated the exquisite sensitivity of mesenchymal stem cells (MSCs) and Dental Pulp Stem Cells (DPSCs) to NK cell mediated cytotoxicity. In addition, we show in this paper that NK cells lyse hESCs and iPS cells significantly. Taken together these results indicated that undifferentiated cells are targets of NK cell cytotoxicity. However, once NK cells lyse a proportion of sensitive targets they lose their cytotoxic function and gain in cytokine secretion capacity (split anergy) which could then support the differentiation of the cells not lysed by the NK cells. Indeed, similar to NK cells cultured with the undifferentiated sensitive tumors and stem cells, the treatment of NK cells with IL-2 and anti-CD16 mAb resulted in the loss of cytotoxicity, gain in IFN-γ secretion and down modulation of CD16 surface receptors. Loss of cytotoxicity and gain in cytokine secretion was also seen when NK cells were cultured with. MSCs and DPSCs in the presence of monocytes.

In vivo physiological relevance of above-mentioned observations could be observed in a subpopulation of NK cells in peripheral blood, uterine and liver NK cells which express low or no CD16 receptors, have decreased capacity to mediate cytotoxicity and is capable of secreting significant amounts of cytokines. In addition, 70% of NK cells become CD16 dim or negative immediately after an allogeneic or autologous bone marrow transplantation. Since NK cells lose their cytotoxic function and gain in cytokine secretion phenotype and down modulate CD16 receptors after their interaction with tumor cells or the stem cells, it is tempting to speculate that in vivo identified CD16− NK cells and in vitro tumor induced CD16− NK cells have similar developmental pathways since they have similar if not identical functional properties:

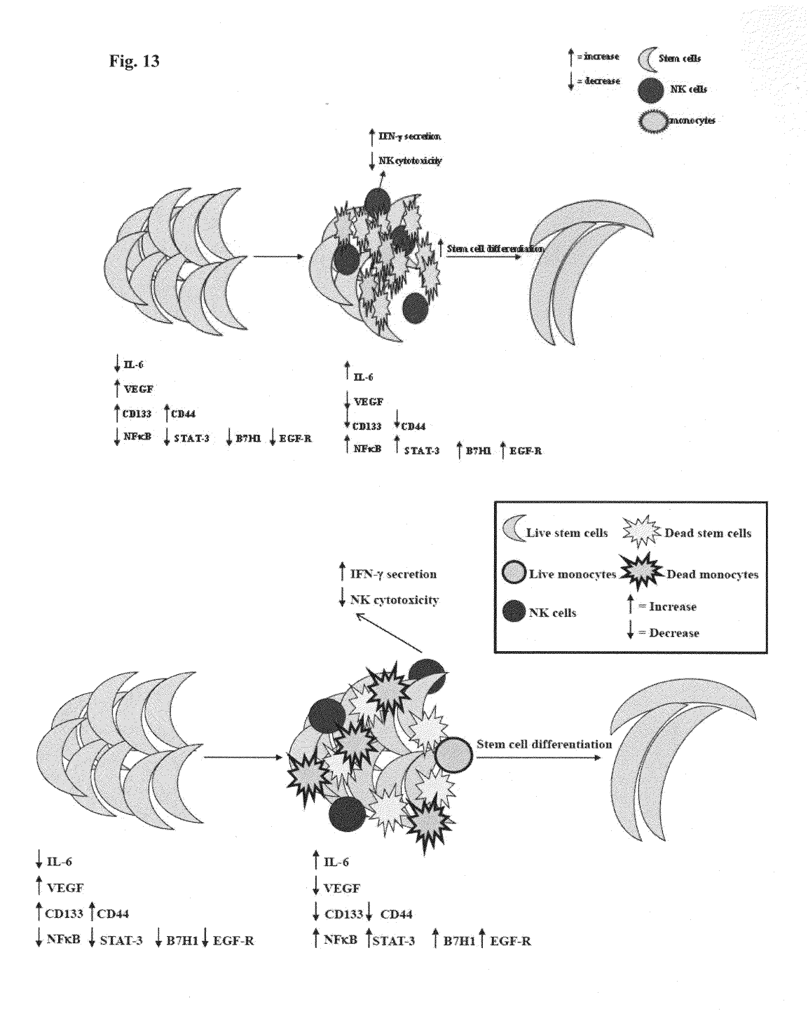

Since undifferentiated cells are targets of NK cells, it is logical that NFκB knock down cells are found to be more susceptible to NK cell mediated cytotoxicity since this process may revert the cells to a relatively less differentiated state and be the cause of activation of NK cells. Indeed, any disturbance in the process of differentiation should provide sensitivity to NK cells since this process is important for modifying the phenotype of NK cells to cytokine secreting cells in order to support differentiation of the remaining competent cells. In this regard knocking COX2 in monocytes may also result in reversion or de-differentiation of the monocytes and the activation of NK cell cytotoxicity. Thus, the degree of differentiation may be predictive of the susceptibility of the cells to NK cell mediated cytotoxicity. In this regard we have also found higher sensitivity of iPS cells to NK cell mediated lysis when compared to parental line from which they were derived. In addition, MSCs not only become resistant to NK cell mediated cytotoxicity after differentiation, but also their level of differentiation increases when they are cultured with the NK cells. As shown here co-culture of NK, monocytes and stem cells are found to result in decreased lysis of stem cells, increased secretion of IFN-γ by the NK cells and elevation in B7H1 surface expression (FIG. 7D and 7E and). Thus, stem cells which survive should exhibit differentiation markers such as increase in NFκB and STAT3 and augmented secretion of GM-CSF, IL-6 and IL-8 after interaction with NK cells and monocytes (FIG. 13).

Based on the results presented herein, NK cells may have two significant functions: one that relates to the removal of stem cells that are either defective or disturbed or in general more in numbers than are needed for the regeneration of damaged tissue. Therefore, the first task is to select stem cells that are competent and are able to achieve the highest ability to differentiate to required cells. The second important task for NK cells is to support the differentiation of the selected cells after altering their phenotype to cytokine secreting cells. This process will not only remove cells that are either infected or transformed, but also it will ensure the regeneration of damaged or defective tissues. Therefore, processes in which suboptimal differentiation and regeneration of the tissues are achieved, a chronic inflammatory process may be established causing continual tissue damage and recruitment of stem cells and NK cells.

The inability of patient NK cells to kill cancer stem cells due to flooding of NK cells by proliferating cancer stem cells and conversion of NK cells to cytokine secreting cells may be one mechanism by which cancer may progress and metastasize. Therefore, there should be two distinct strategies by the NK cells to eliminate tumors, one that targets stem cells and the other that targets differentiated cells. This can be achieved in oral cancer patients by the use of EGFR antibody since this antibody targets the differentiated oral tumors whereas stem cells should be eliminated by the NK cells. However, since a great majority of patient NK cells have modified their phenotype to support differentiation of the cells, they may not be effective in eliminating the cancer stem cells. Therefore, cancer stem cells may accumulate and eventually result in the demise of the patient. These patients can benefit from repeated allogeneic NK cell transplantation for elimination of cancer stem cells.

TABLE 1
UCLA-OSCSCs similar to HEp2-IκB_{(S32AS36A)} tumor
cells secreted no or lower levels of GM-CSF, IL-6 and IL-8.

| | GM-CSF | IL-6 | | IL-8 |
|---|---|---|---|---|
| | pg/ml (MFI*) | pg/ml (MFI) | | pg/ml (MFI) |
| HEp2-vec | 0 ± 0 (30) | 20.6 ± 1 | (565) | 685 ± 20 (1390) |
| HEp2-IκB_{(S32AS36A)} | 0 ± 0 (29) | 1.5 ± 0 | (67) | 17 ± 0 (453) |
| UCLA-OSCCs | 19.8 ± 2 (79) | 58.4 ± 3 | (1554) | 906.3 ± 50 (7583) |
| UCLA-OSCSCs | 0 ± 0 (32) | 0 ± 0 | (11) | 245.2 ± 12 (3247) |

HEp2-vec, HEp2-IkB_{(S32AS36A)}, UCLA-OSCCs, and UCLA-OSCSCs were cultured at 1 × 10⁵ cells/ml and the constitutive levels of secreted GM-CSF, IL-6, and IL-8 were determined using multiplex ELISA array kit. The concentrations of secreted cytokines were determined using the standard curve for each cytokine.
*Mean fluorescence intensity (MFI). One of three representative experiments is shown.

TABLE 2
Increased ratios of IL-6 to IFN-γ secretion in NK resistant
UCLA-OSCCs when compared to NK sensitive UCLA-OSCSCs.

| | +/−Immune | GM-CSF | IL-8 | VEGF | IL-6 | IFN-γ | Ratio IL-6/ |
|---|---|---|---|---|---|---|---|
| Tumor cells | cells | pg/ml | pg/ml | pg/ml | pg/ml | pg/ml | IFN-γ |
| UCLA-OSCCs | −NK | 20 | 438.4 | 620.4 | 126 | 0.8 | — |
| UCLA-OSCCs | +NK (−IL-2) | 148.8 | 723.2 | 784 | 215 | 1 | 215 |
| UCLA-OSCCs | +NK (+IL-2) | 565.8 | 282.2 | 145.5 | 179 | 820 | 0.22 |
| UCLA-OSCSCs | −NK | 0.1 | 23.3 | 1745 | 13 | 1 | — |
| UCLA-OSCSCs | +NK (−IL-2) | 25 | 66.7 | 1256 | 65 | 1 | 12.5 |
| UCLA-OSCSCs | +NK (+IL-2) | 1068.9 | 12.5 | 158 | 12 | 1730.6 | 0.007 |
| No tumors | +NK (−IL-2) | 0.8 | 0 | 0.4 | 11 | 0.6 | 18 |
| No tumors | +NK (+IL-2) | 403.2 | 3.14 | 8.6 | 13 | 290 | 0.44 |

NK cells (1 × 10⁶/ml) were left untreated or treated with IL-2 (1000 units/ml) for 12-24 hours before NK cells (1 × 10⁵/ml) were added to primary oral tumors at an effector to target ratio of 1:1. Tumor cells were each cultured alone or in combination with NK cells as indicated in the table and the supernatants were removed from the cultures after an overnight incubation. The levels of cytokine secretion were determined using antibody coated multiplex microbead immunoassay. For simplification of the table standard deviations are not included and they ranged from 0% to a maximum of 5% of the amount obtained for each cytokine. One of three representative experiments is shown.

### Example 2

### Increased NK cell cytotoxicity against UCLA-OSCSCs but not those of UCLA-OSCCs

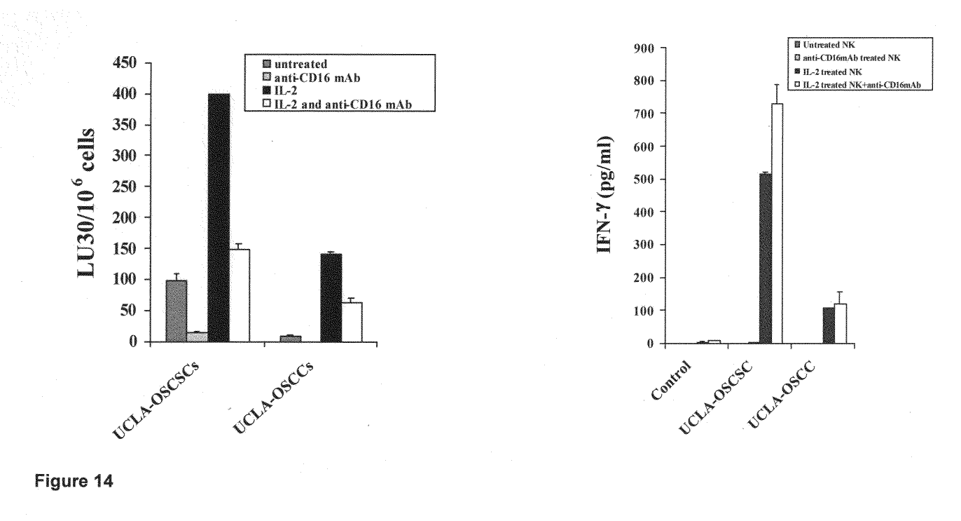

UCLA-OSCC and UCLA-OSCSCs were tested for their sensitivity or resistance to NK cell mediated cytotoxicity. The cytotoxic activities of IL-2 treated PBMCs (FIG. 14A) and NK cells (FIG. 14B) were significantly higher against UCLA-OSCSCs cells when compared to UCLA-OSCCs. Untreated PBMCs or NK cells lysed UCLA-OSCSCs tumors significantly more than UCLA-OSCCs (FIG. 14B). However, the levels of lysis by untreated NK cells were considerably lower than that obtained by IL-2 treated PBMCs or NK cells (FIG. 14A and 14B). Treatment of PBMCs or NK cells with anti-CD16 mAb decreased cytotoxicity significantly against both tumor types, however, the levels of lysis by the NK cells remained higher against UCLA-OSCSCs in all the NK samples tested (FIG. 14). IL-2 treated NK cells co-cultured with UCLA-OSCSCs oral tumor cells exhibited higher expression of CD69 activation antigen when compared to those co-cultured with UCLA-OSCC oral tumors.

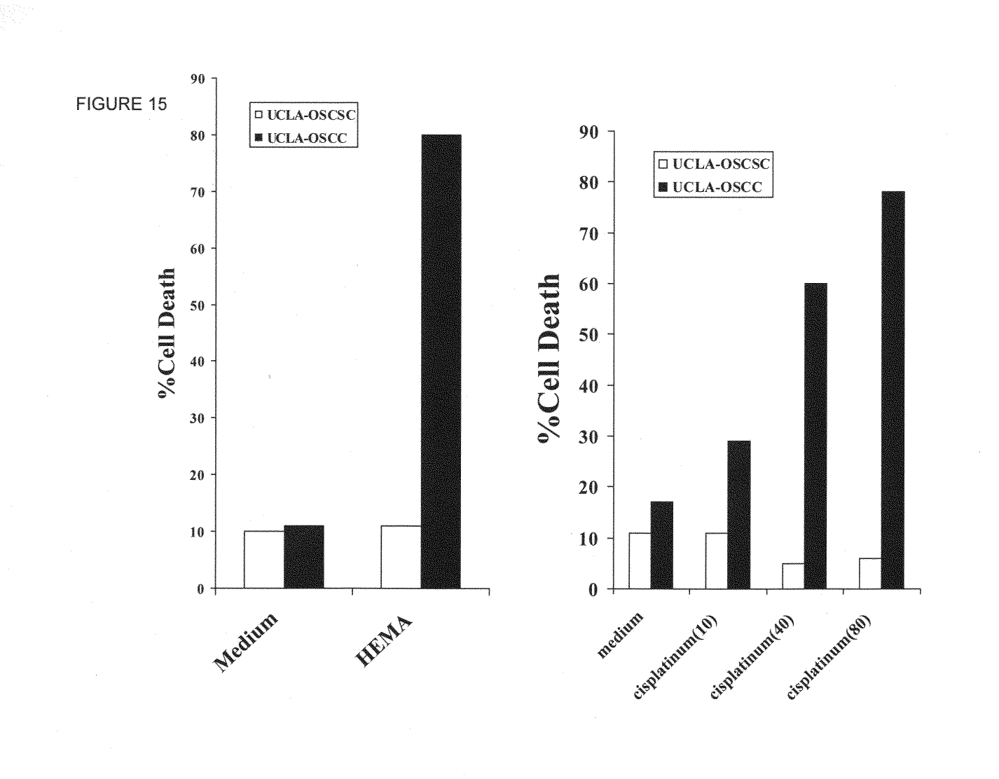

OSCSCs and OSCCs were treated with HEMA (1:600) and the chemotherapeutic drug cisplatinum at 10 μg/ml, 40 μg/ml and 80 μg/ml. After an overnight incubation the levels of cell death in each tumor type was determined using propidium iodide staining. As shown in FIG. 15 both HEMA and cisplatinum lysed OSCCs significantly; however, OSCSC were significantly more resistant to toxicity mediated by these drugs.

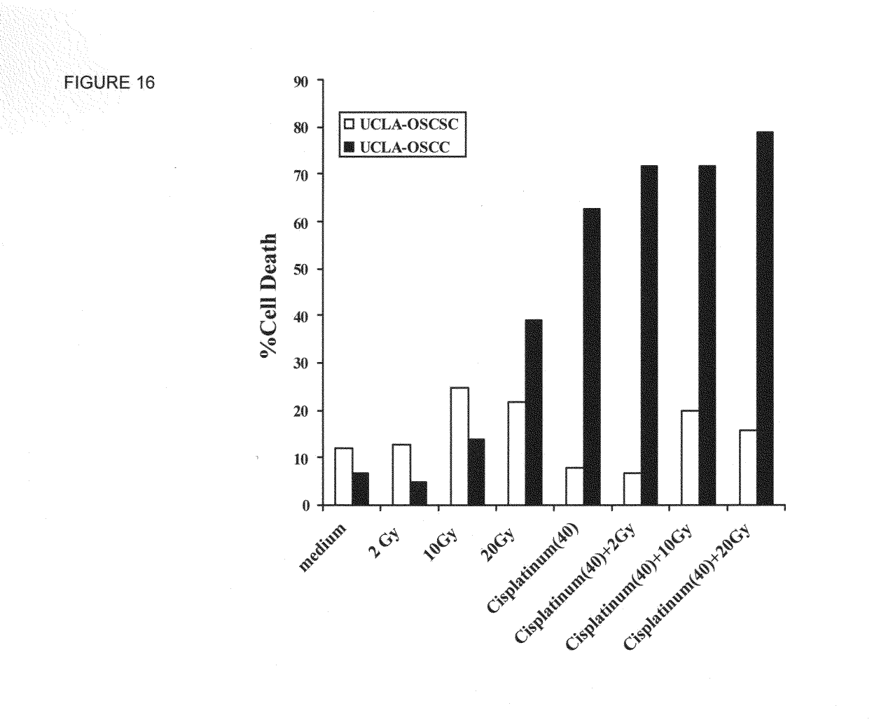

OSCSCs and OSCCs were irradiated at 2 Gy, 10 Gy and 20 Gy and left untreated or treated with chemotherapeutic drug cisplatinum at 40 μg/ml. After an overnight incubation the levels of cell death in each tumor type was determined using propidium iodide staining. As shown in FIG. 16 both radiation and cisplatinum treatment lysed OSCCs significantly; however, OSCSC were significantly more resistant to toxicity mediated by radiation and cisplatinum.

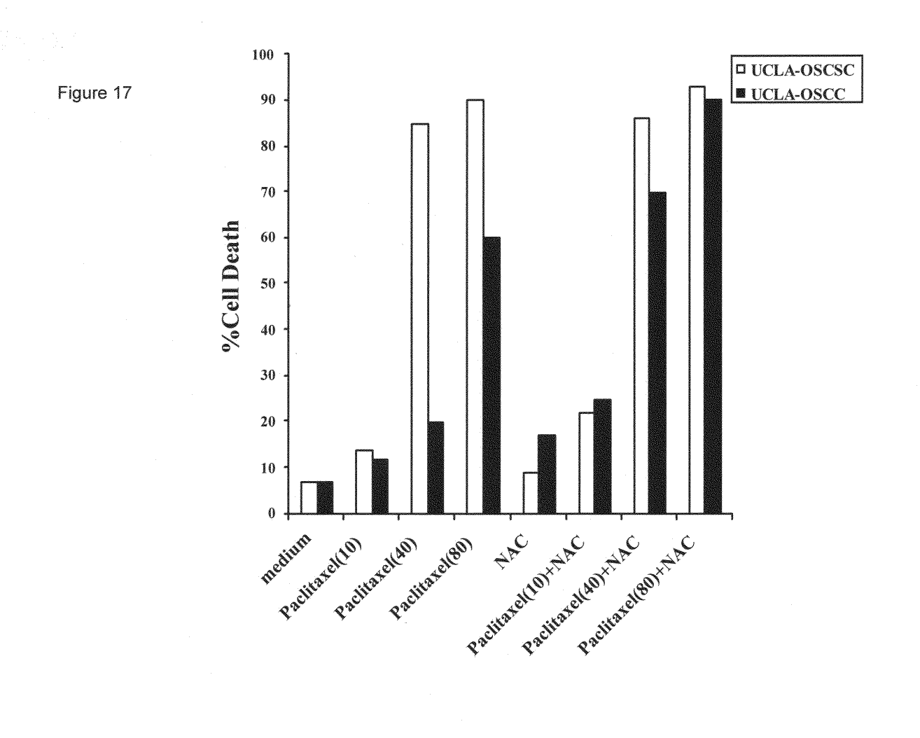

OSCSC and OSCCs were treated with Paclitaxel at 10 μg/ml, 40 μg/ml and 80 μg/ml in the presence and absence of NAC (20 mM) and after an overnight incubation the levels of cell death were determined using propidium iodide staining. As shown in FIG. 17 paclitaxel lysed both cell types. In addition, NAC synergized with paclitaxel to lyse cancer stem cells. Therefore, to eliminate cancer stem cells two potential treatments will be effective. 1-using NK cells as an immunotherapeutic strategy and 2-the use of Paclitaxel in combination with NAC.

### Example 3

### Significant lysis of Embryonic Stem Cells (hESCs), Induced Plueripotent Stem Cells (iPS), Dental Pulp Stem Cells (DPSCs), and Mesenchymal Stem Cells (MSCs) by untreated or IL-2 treated NK cells

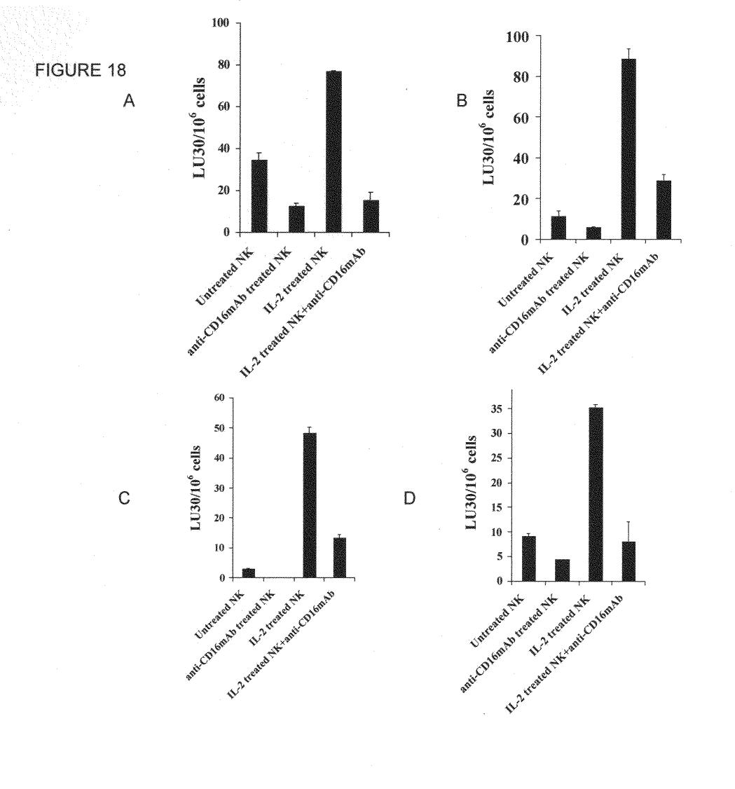

Highly purified human NK cells were cultured with and without IL-2 for 12-24 hours before they were added to ⁵¹Cr labeled hESCs (FIG. 18A), iPSCs (FIG. 18B), DPSCs (FIG. 18C) and MSCs (FIG. 18D). Addition of untreated NK cells had lower cytotoxicity against different populations of stem cells whereas activation with IL-2 increased cytotoxicity against all stem cell populations significantly (p<0.05). Therefore, human stem cells are greatly lysed by the NK cells.

Lysis of hESCs, iPSCs, DPSCs, and MSCs by untreated and IL-2 treated NK cells is inhibited by anti-CD16 antibody treatment, however, the same treatment induced significant secretion of IFN-γ by the NK cells in the presence and absence of stem cells.

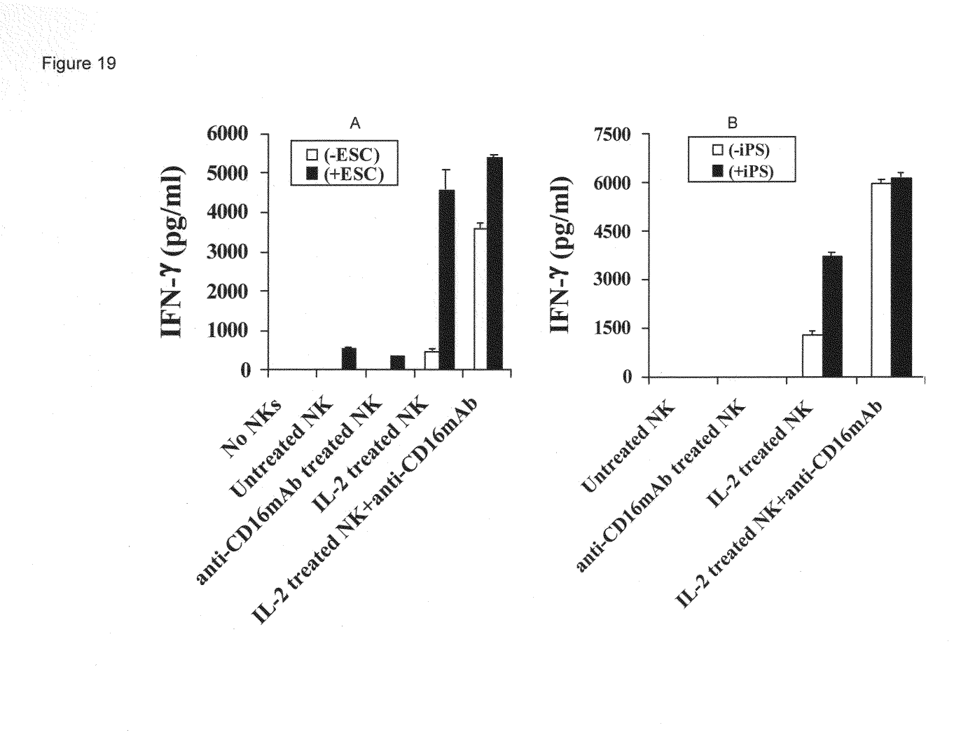

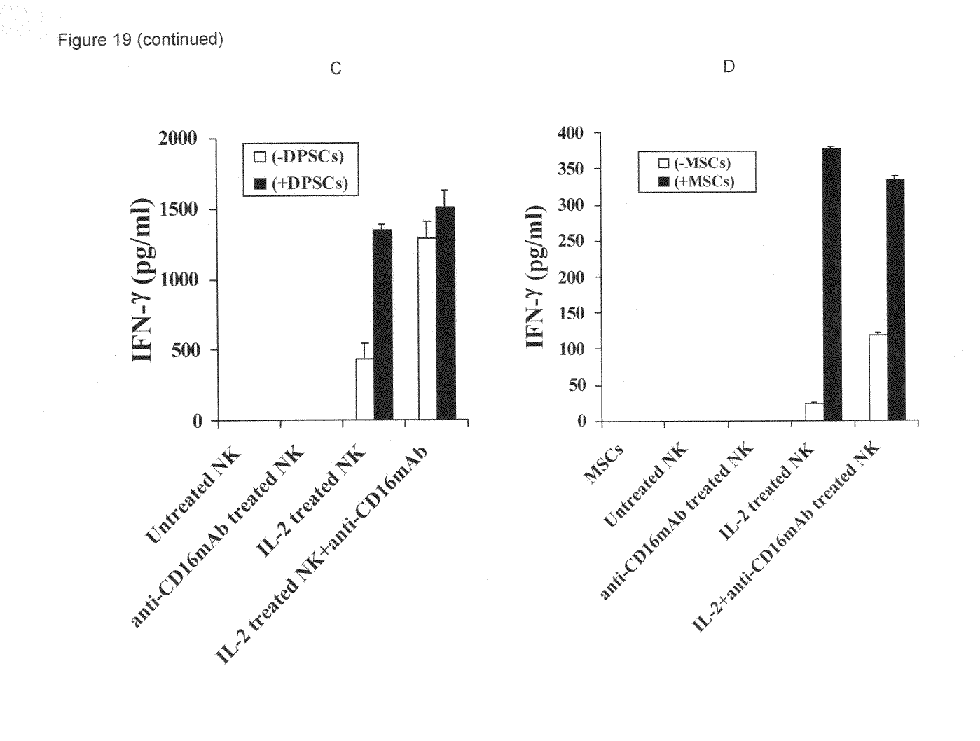

As shown in a number of previous studies anti-CD16 mAb treatment induced anergy in a great majority of the NK cells as well as it induced death in a subset of NK cells, thereby inhibiting NK cell cytotoxicity against different populations of stem cells (p<0.05). Addition of the combination of IL-2 and anti-CD16 treatment also induced anergy and NK cell death and inhibited NK cell cytotoxicity against stem cells when compared to IL-2 activated NK cells (p<0.05). Untreated or anti-CD16 mAb treated NK cells did not secrete IFN-γ when co-cultured with any of the stem cell populations, however, both IL-2 treated and IL-2 in combination with anti-CD16 mAb treated NK cells in the presence and absence of stem cells secreted significant levels of IFN-γ (p<0.05) (FIG. 19A-19D). Indeed, stem cells triggered significant secretion of IFN-γ from IL-2 treated NK cells when compared to IL-2 treated NK cells in the absence of stem cells. In addition, there was a synergistic induction of IFN-γ secretion in IL-2 and anti-CD16 mAb treated NK cells in the absence of stem cells, and the levels either remained the same or exceeded those in the absence of stem cells when IL-2 and anti-CD16 mAb treated NK cells were cultured with stem cells (FIG. 19A-19D). There was a direct correlation between secretion of bFGF by stem cells and cytotoxicity by IL-2 and IL-2+ anti-CD16 treated NK cells.

Lysis of MSCs by untreated and IL-2 treated NK cells is inhibited by monocytes, however, the addition of monocytes induced significant secretion of IFN-γ by the NK cells in the presence and absence of stem cells. Monocytes were purified from PBMCs and irradiated as indicated in the Material and Methods section. MSCs were co-cultured with irradiated monocytes for 24-48 hours before they were labeled with ⁵¹Cr and used in the cytotoxicity assays against NK cells. NK cells were left untreated or pre-treated with anti-CD16 antibody, and/or IL-2 for 24-48 hours before they were used in the cytotoxicity assays against MSCs. The addition of monocytes to MSCs significantly protected the MSCs from NK cell mediated cytotoxicity (p<0.05). Significant inhibition of NK cell cytotoxicity by monocytes could be observed against untreated and IL-2 treated NK samples (p<0.05). Monocytes also increased the levels of alkaline phosphatase staining in MSCs and prevented decrease in alkaline phosphatase expression induced by IL-2 activated NK cells. Untreated or anti-CD16 antibody treated irradiated monocytes did not mediate cytotoxicity against MSCs. Overall, these experiments indicated that monocytes protect MSCs against NK cell mediated lysis.

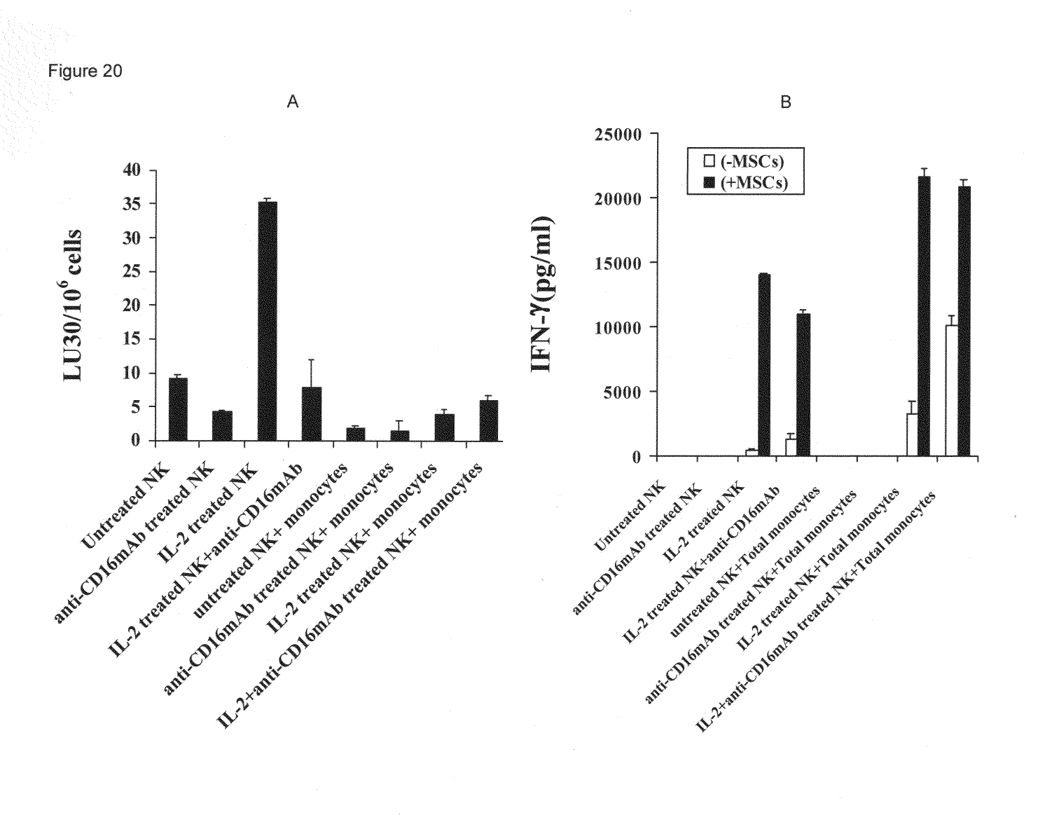

As expected IL-2 treated NK cells secreted moderate amounts of IFN-γ which were synergistically increased when co-cultured in the presence of MSCs (p<0.05) (FIG. 20A-B). The addition of anti-CD16 mAb in combination with IL-2 to NK cells in the absence of MSCs increased secretion of IFN-γ, when compared to IL-2 alone treated NK cells in the absence of MSCs. IFN-γ secreted levels remained similar between IL-2 alone and IL-2 and anti-CD16 mAb treated NK cells cultured with MSCs. Monocytes added to IL-2 or IL-2 and anti-CD16 antibody treated NK cells in the absence of MSCs or those in the presence of MSCs, synergistically increased the levels of secreted IFN-γ (p<0.05). However, the highest increase in IFN-γ release was seen when monocytes were added to IL-2 or IL-2 and anti-CD16 mAb treated NK cells with MSCs. These results indicated that monocytes increased IFN-γ in co-cultures with MSCs, and further synergized with IL-2 or IL-2 and anti-CD16 mAb treated NK samples to increase the release of IFN-γ in the co-cultures of NKs and MSCs. Similar results were obtained when NK cells were co-cultured with monocytes and DPSCs.

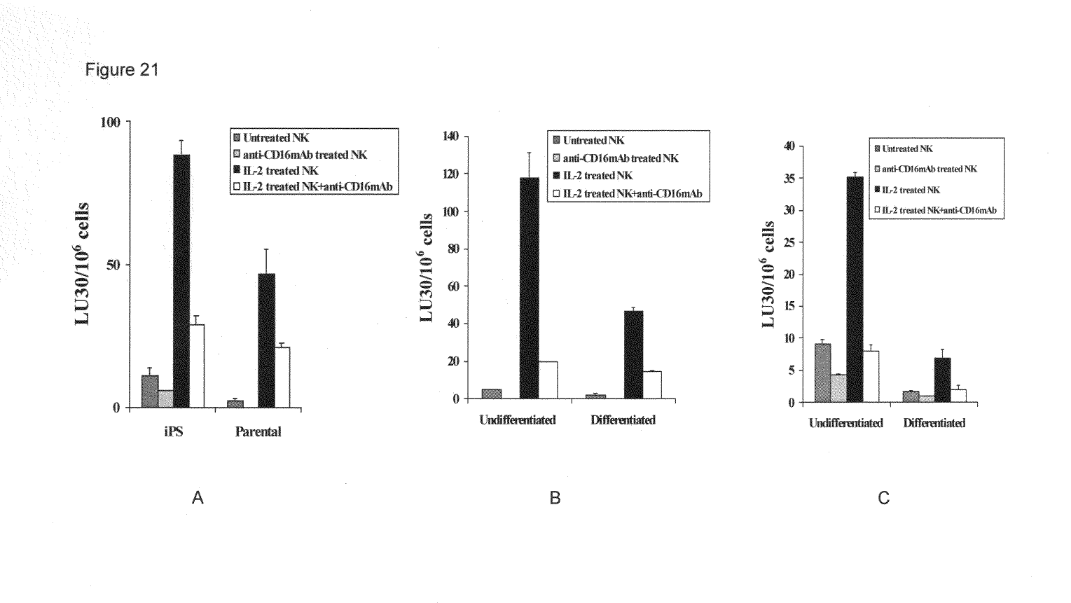

Differentiated DPSCs are more resistant to NK cell mediated cytotoxicity. DPSCs were differentiated to odontoblasts by the addition of b-glycerophosphate, ascorbic acid and dexamethasone as reported previously, and NK cell cytotoxicity were determined against both the differentiated and undifferentiated DPSCs. As shown in FIG. 21, significantly less NK cell cytotoxicity as well as IFN-γ secretion could be obtained against differentiated DPSCs by untreated, IL-2 treated and IL-2 plus anti-CD16 mAb treated NK cells when compared to undifferentiated DPSCs. In addition, significantly less NK cell cytotoxicity could be seen against passage 10 undifferentiated DPSCs when compared to passage 3 undifferentiated DPSCs. Therefore, a stepwise decrease in NK cell cytotoxicity could be observed depending on the stage of the differentiation of DPSCs.

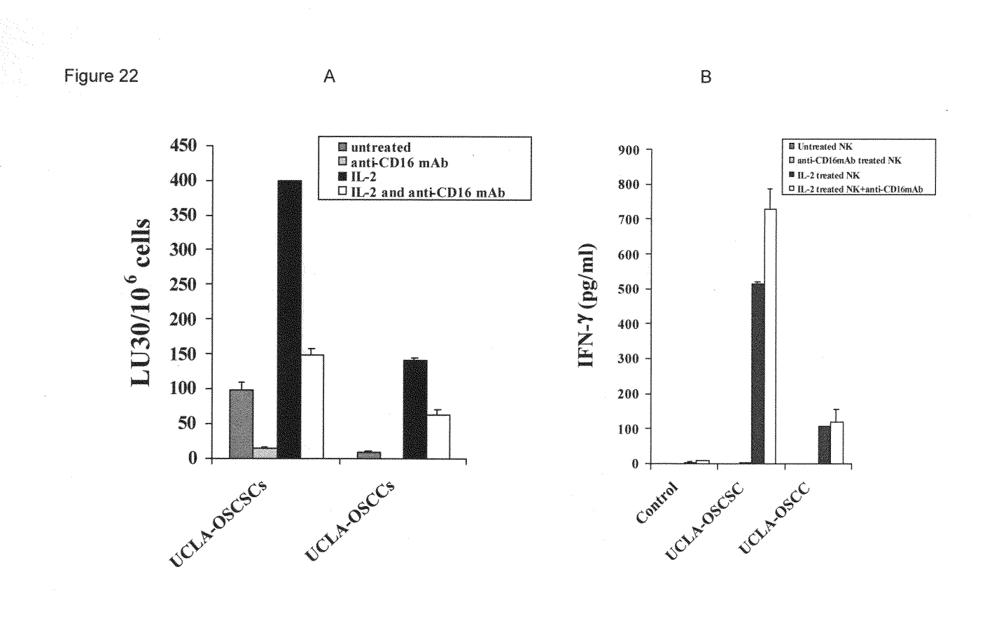

Increased NK cell cytotoxicity against UCLA-OSCSCs but not those of UCLA-OSCCs. UCLA-OSCC and UCLA-OSCSCs were tested for their sensitivity or resistance to NK cell mediated cytotoxicity. The cytotoxic activities of IL-2 treated PBMCs (FIG. 22A) and NK cells (FIG. 22B) were significantly higher against UCLA-OSCSCs cells when compared to UCLA-OSCCs. Untreated PBMCs or NK cells lysed UCLA-OSCSCs tumors significantly more than UCLA-OSCCs. However, the levels of lysis by untreated NK cells were considerably lower than that obtained by IL-2 treated PBMCs or NK cells. Treatment of PBMCs or NK cells with anti-CD16 mAb decreased cytotoxicity significantly against both tumor types, however, the levels of lysis by the NK cells remained higher against UCLA-OSCSCs in all the NK samples tested. IL-2 treated NK cells co-cultured with UCLA-OSCCs oral tumor cells exhibited higher expression of CD69 activation antigen when compared to those co-cultured with UCLA-OSCC oral tumors.

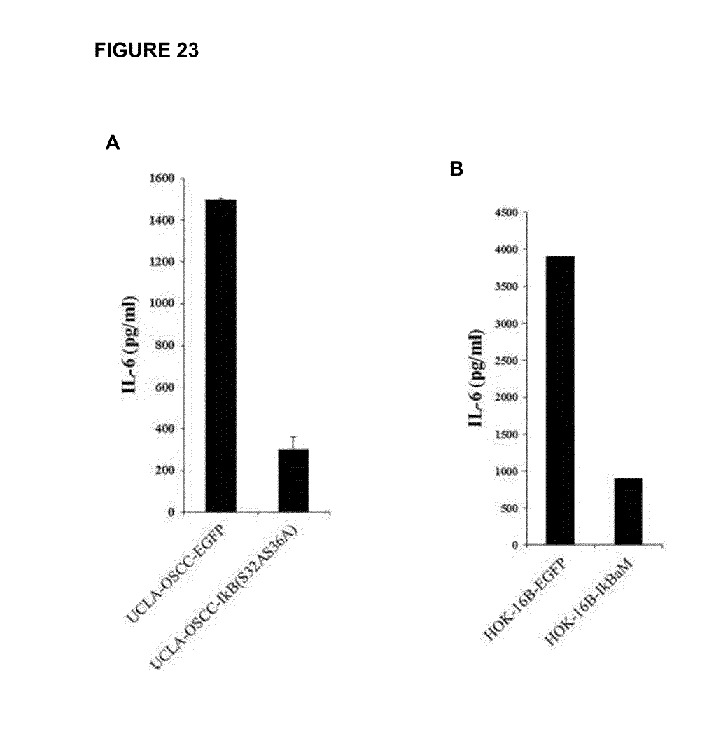

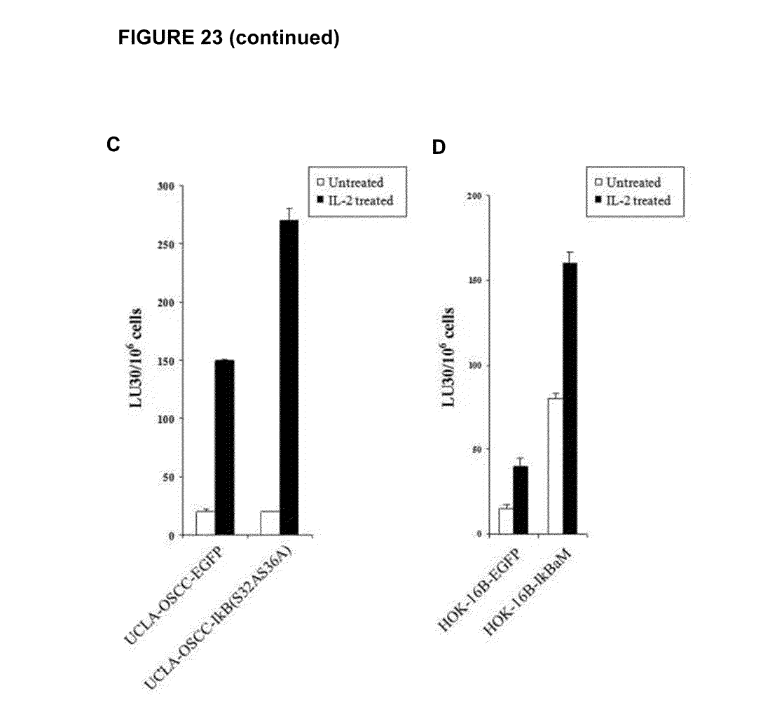

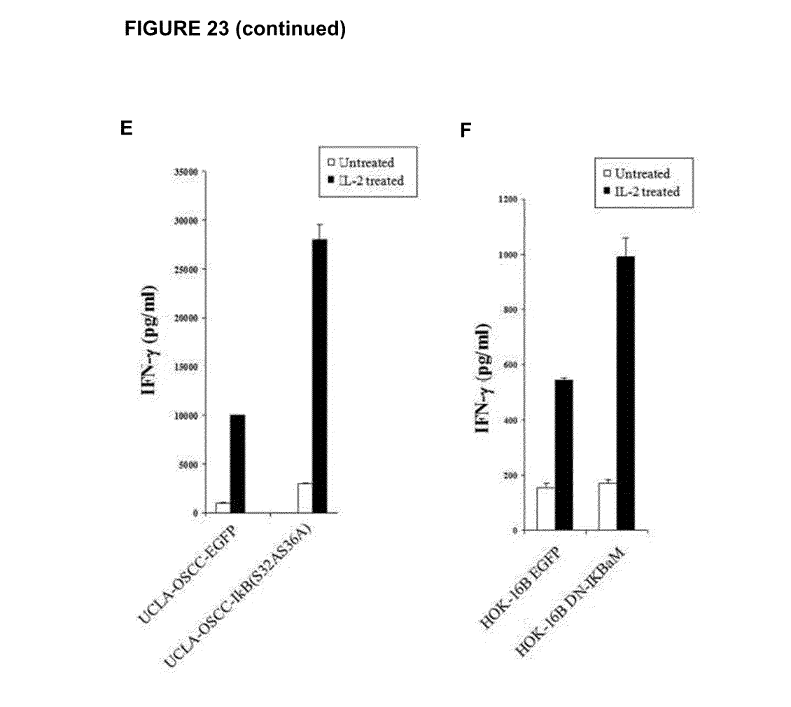

Blocking NFκB in UCLA-OSCCs and HOK-16B oral epithelial cells lowered IL-6 to IFN-γ ratios and increased their sensitivity to NK cell mediated cytotoxicity. Immortalized but non-tumorigenic oral keratinocytes (HOK-16B) or UCLA-OSCCs were transduced with EGFP alone, IκBαM or IκB_{(S32AS36A)}super-repressor retroviral vectors and sorted for high GFP expressing cells using flow cytometry. The inhibition of NFκB by the IκBαM or IκB_{(S32AS36A)}super-repressor retroviral vectors in HOK-16B or UCLA-OSCC was confirmed by measuring NFκB activity using luciferase reporter assay (data not shown). IκBαM or IκB_{(S32AS36A)}super-repressor transduced HOK-16B (FIG. 23A) or UCLA-OSCC (FIG. 23B) cells secreted substantially lower levels of IL-6 when compared to EGFP transduced cells. NFκB knock down UCLA-OSCCs and HOK-16B cells did not exhibit elevated levels of cell death when assessed by flow cytometric analysis of . Annexin V and PI stained cells. IL-2 treated NK cells lysed NFκB knock down OSCCs (FIG. 23C) and HOK-16B (FIG. 23D) cells significantly more than EGFP transfected cells. In addition, IL-2 activated NK cells secreted lower levels of IL-6 (data not shown) but higher amounts of IFN-γ in supernatants obtained from the co-cultures of NK cells with NFκB knock down UCLA-OSCCs (FIG. 23E) and HOK-16B (FIG. 23F) cells as compared to EGFP transduced cells. Similar NK cell response patterns were obtained when NFκB was inhibited in HEp2 cells.

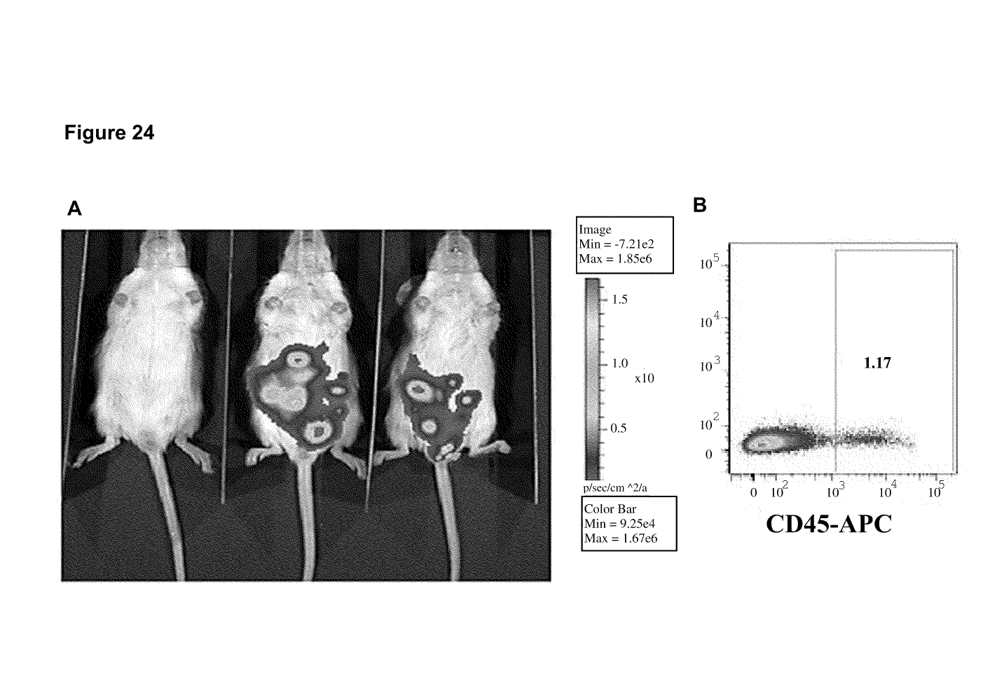

Bioluminescent tracking of engrafted HESC in RAG2-/-gc-/- Mice. Human ES cells (HESC; Hues9) transduced with lentiviral luciferase GFP for 24 hours were transplanted intrahepatically into neonatal RAG2-/-γc-/- mice followed by imaging (IVIS 200) at 4, 6 and 8 weeks (FIG. 24A). Mice were also monitored for teratoma formation. Human CD34+CD38-cells (50,000 per mouse) from a patient with poor risk AML were transplanted intrahepatically into neonatal RAG2-/-γc-/- mice leading to FACS detectable human CD45 (X-axis) engraftment within 8 weeks of transplantation (FIG. 24B).

### Example 3

### Effect of NK cells and the combination of NAC and Paclitaxel on Oral, Pancreatic, Lung, Prostate and Breast Tumors

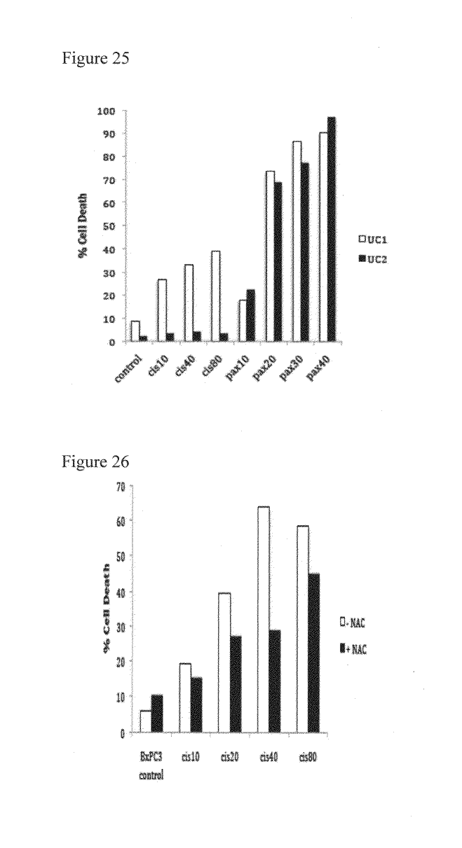

Dose dependent effect of cisplatin and Paclitaxel on two oral tumors (UC-1 is OSCCs, differentiated oral tumor whereas UC2 is OSCSCs, stem-like oral tumors). Cisplatin has increased effect on differentiated OSCCs whereas it does not kill OSCSCs. Paclitaxel affects both cell types similarly (FIG. 25)

Dose dependent increase in cisplatin mediated killing of pancreatic cells (BXPC3). NAC inhibits cisplatin mediated killing of pancreatic cells (BXPC3) (FIG. 26).

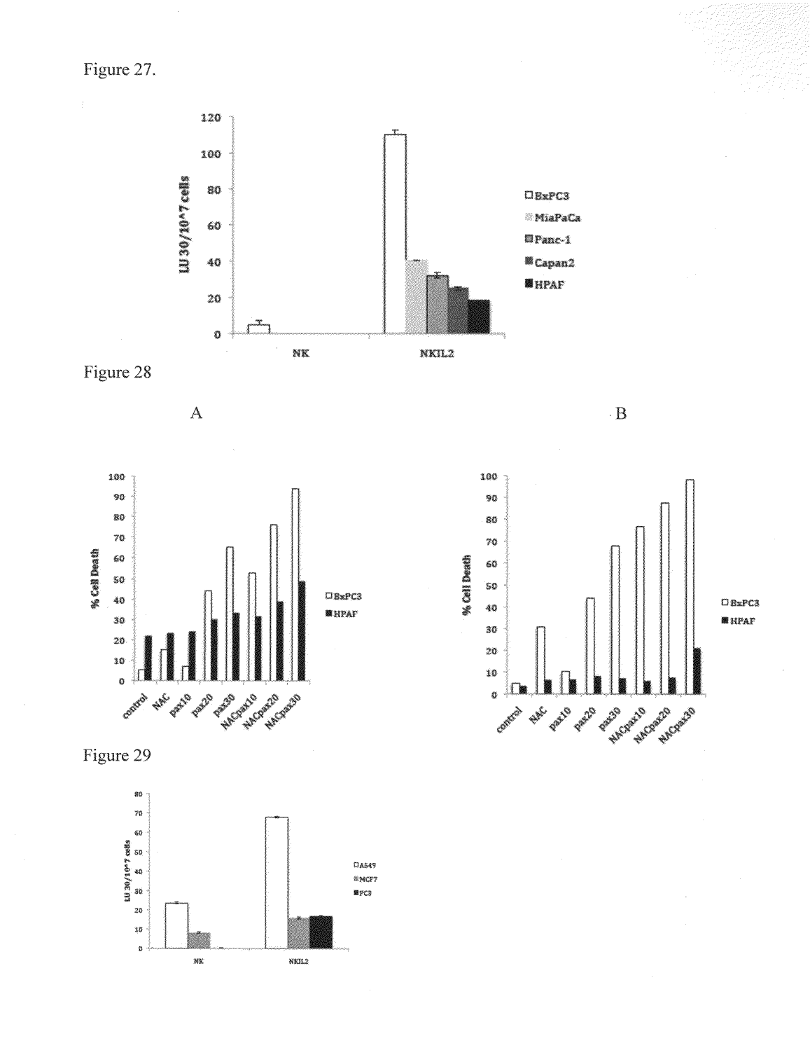

BxPC3 is less differentiated, and a more stem-like pancreatic tumor line based on surface analysis, and more sensitive to NK cell mediated cytotoxicity as reported previously for oral tumors (Tseng et al, 2010) whereas HPAF is more of a differentiated pancreatic cell type and much more resistant to NK cell mediated cytotoxicity. The two cell types were selected from 5 pancreatic cell lines based on the highest and the lowest sensitivity to NK cell mediated lysis (FIG. 27).

Dose dependent synergistic induction of cell death by NAC and Paclitaxel in BXPC3 pancreatic cells, please note HPAF is more resistant to Paclitaxel and NAC mediated cell death than BXPC3 (FIG. 28A-28B).

The cytotoxic function of purified NK cells were assessed against lung tumors (A549), Breast tumors (MCF7) and Prostate tumors (PC3). A549 was found to be more sensitive to both untreated and IL-2 treated NK cell mediated cytotoxicity than MCF7 or PC3.

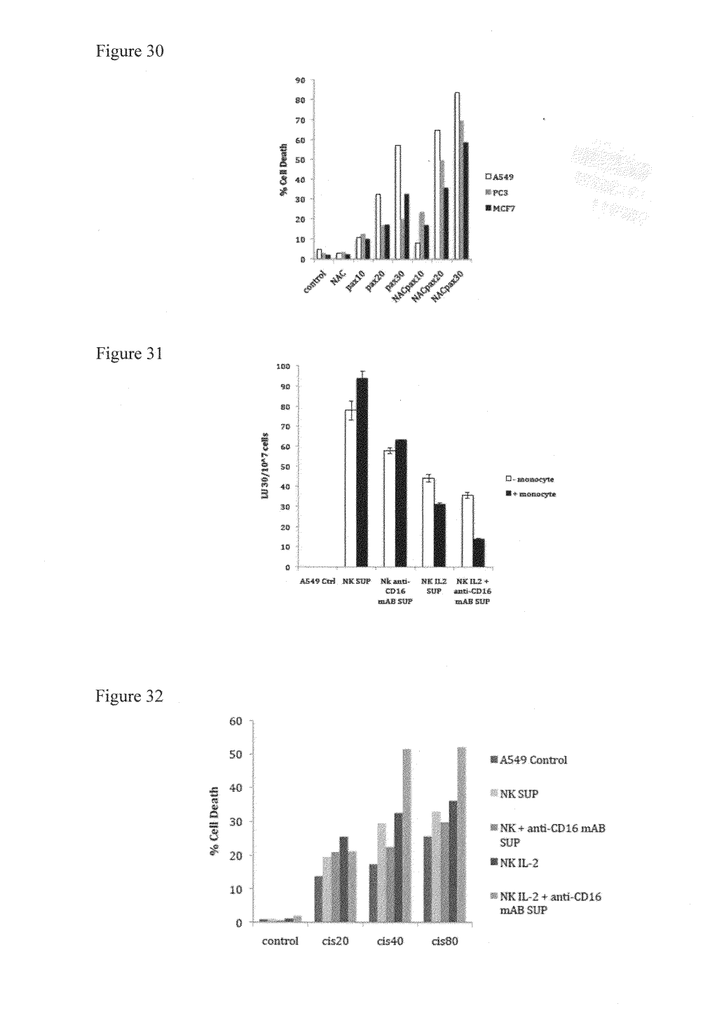

Dose dependent synergistic effect of NAC and Paclitaxel on lung (A549), prostate (PC3) and breast (MCF7) tumors (FIG. 30).

A₅₄₉ lung tumors were cultured with supernatants removed from untreated NK, anti-CD16 mAb treated NKs, IL-2 treated NKs and IL2 in combination of anti-CD16 mAb treated NK cells in the presence and absence of monocytes. NK treatments were carried out for 24 hours before the supernatants were removed and used to treat A549 lung tumors for 5 days. As shown in the figure treatment of A549 with supernatants from the anergized NK cells (IL-2+anti-CD16 mAb) for 5 days caused the most resistant to NK cell mediated lysis when exposed to IL-2 treated NK cells. Please note the killing of A549 without the NK supernatants are not shown in this figure. Anti-CD16 mAb or IL-2 treated NK cell supernatants also caused resistance in A549 cells when compared to A549 treated with supernatants removed from untreated NK cells. Therefore, these results indicated that anergized NK cells are important for the differentiation and resistance of lung tumors. (FIG. 31)

Similarly, A549 lung tumors were cultured with supernatants removed from untreated NK, anti-CD16 mAb treated NKs, IL-2 treated NKs and IL2 in combination of anti-CD16 mAb treated NK cells in the presence and absence of monocytes. NK treatments were carried out for 24 hours before the supernatants were removed and used to treat A549 lung tumors for 5 days. All the NK supernatant treated lung tumors were then washed and treated with different concentrations of Cisplatin as shown in the figure and incubated overnight before they were stained with propidium iodide to determine cell death in each sample. As shown in the figure treatment of A549 with supernatants from the anergized NK cells (IL-2+anti-CD16 mAb) for 5 days and then exposed to Cisplatin resulted in the highest induction of cell death by Cisplatin. Untreated NK sup or those treated with anti-CD16 mAb or IL-2 treated NK cell supernatants also caused low to moderate increases in cisplatin mediated death of A549 cells when compared to A549 with media alone without NK cell supernatants. Therefore, these results indicated that anergized NK cell supernatant can not only differentiate the lung cells and cause resistance against NK cell mediated cytotoxicity but it can also make the lung tumors more sensitive to chemotherapeutic drugs such as cisplatin mediated cells death. (FIG. 32)

What is Claimed is:

1. A method for depleting cancer stem cells at a tumor site in an individual, the method comprising:
depleting effector cells at the site; and
administering a composition of activated NK cells at a dose effective to deplete the cancer stem cells.

2. The method of claim 1, wherein the cancer stem cells are carcinoma stem cells.

3. The method of claim 2, wherein the carcinoma is a squamous cell carcinoma.

4. The method of claim 3, wherein said cancer stem cells are identified by expression of one or both of CD44 and CD133.

5. The method of claim 1, wherein effector cells are depleted by a dose of radiation sufficient to substantially eliminate monocytes in the tumor microenvironment.

6. The method of claim 1, wherein effector cells are depleted by a dose of chemotherapy sufficient to substantially eliminate monocytes in the tumor microenvironment.

7. The method of claim 1, wherein the NK cells are autologous.

8. The method of claim 1, wherein the NK cells are allogeneic.

9. The method of claim 1, wherein the NK cells are enriched from a peripheral blood sample and activated by culture in the presence of IL-2, optionally with additional IL12 or interferon alpha.

10. The method of claim 1, wherein the NK cells are injected at the site of the tumor.

11. The method of claim 1, further comprising administering to the individual an effective dose of a taxane and N-acetylcysteine.

12. The method of claim 11, wherein the taxane is paclitaxel.

13. The method of claim 1, further comprising administering to the individual a chemotherapeutic agent that depletes differentiated cancer cells.

14. A method for depleting cancer stem cells at a tumor site in an individual, the method comprising:
administering a combination of an effective dose of a taxane and N-acetylcysteine.

15. The method of claim 14, wherein the taxane is paclitaxel.

16. The method of claim 14, wherein the cancer stem cells are carcinoma stem cells.

17. The method of claim 16, wherein the carcinoma is a squamous cell carcinoma.

18. A method for establishing tolerance to a stem cell graft, the method comprising administering an anti-CD16 antibody to tolerize NK cells to support differentiation of stem cells.

19. The method of claim 16, wherein NK cells are treated with IL-2 and/or IL-12, or IFN-α in the presence of anti-CD16 antibody to induce split anergy.

20. A method of claim 18 for establishing tolerance to a stem cell graft, the method comprising introducing stem cells mixed with monocytes for engraftment.
